(19) United States
(12) Patent Application Publication
(10) Pub. No.: US 2025/0360022 A1
(43) Pub. Date: Nov. 27, 2025

(54) ADJUSTING FEEDBACK SYSTEM FOR AN INTRAORAL DEVICE

(71) Applicant: RESMED PTY LTD, Bella Vista (AU)

(72) Inventors: Michael Christopher Hogg, Sydney (AU); Angelene Marie Ozolins, Sydney (AU); Bernadette Pudadera, Sydney (AU); Alwin Hui, Sydney (AU); Hugh Francis Stewart Thomas, Sydney (AU); Keira Moore, Sydney (AU); Michiel Kooij, Sydney (AU); Bao Hui Lee, Singapore (SG); Michael Broderick, Dublin (IE); Roxana Tiron, Dublin (IE); Sam Coffey, Dublin (IE); Graeme Alexander Lyon, Dublin (IE); Niall Andrew Fox, Dublin (IE)

(21) Appl. No.: 19/216,123

(22) Filed: May 22, 2025

(30) Foreign Application Priority Data
May 22, 2024 (AU) 2024901510

(51) Int. Cl. A61F 5/56; A61B 5/00

(52) U.S. Cl. CPC A61F 5/566; A61B 5/4818; A61B 5/4833; A61F 2005/563

(57) ABSTRACT

An intraoral device system includes an intraoral device; an upper splint structured to engage with at least a portion of one or more teeth on a maxilla of a patient; a lower splint structured to engage with at least a portion of one or more teeth on a mandible of the patient; a drive system for moving the lower splint with respect to the upper splint; and an adjusting feedback system. The adjusting feedback system can comprise a memory storing machine-readable instructions; and a control system including one or more processors configured to execute the machine-readable instructions. The machine readable instructions can cause the control system to: receive sensor data from one or more sensors associated with the patient; process the sensor data to determine a respiratory event likelihood parameter of the patient; and output control instructions to the intraoral device that operate the drive system to move the lower splint with respect to the upper splint responsive to the determined respiratory event likelihood parameter.

### CROSS-REFERENCE TO RELATED APPLICATIONS

This application claims the benefit of and priority to Australian Patent Application No. 2024901510, filed on May 22, 2024, which is incorporated by reference herein in its entirety.

### 1.1 FIELD OF THE TECHNOLOGY

The present technology relates to an intraoral device for preventing and/or treating snoring and/or obstructive sleep apnea. In particular, the present technology relates to a mandibular repositioning device (MRD) or Mandibular advancement device (MAD) for treating and/or preventing snoring and/or obstructive sleep apnea.

### 1.2 DESCRIPTION OF THE RELATED ART

### 1.2.1 Human Respiratory System and its Disorders

The respiratory system of the body facilitates gas exchange. The nose and mouth form the entrance to the airways of a patient.

The airways include a series of branching tubes, which become narrower, shorter and more numerous as they penetrate deeper into the lung. The prime function of the lung is gas exchange, allowing oxygen to move from the inhaled air into the venous blood and carbon dioxide to move in the opposite direction. The trachea divides into right and left main bronchi, which further divide eventually into terminal bronchioles. The bronchi make up the conducting airways, and do not take part in gas exchange. Further divisions of the airways lead to the respiratory bronchioles, and eventually to the alveoli. The alveolated region of the lung is where the gas exchange takes place, and is referred to as the respiratory zone. See “Respiratory Physiology”, by John B. West, Lippincott Williams & Wilkins, 9th edition published 2012.

A range of respiratory disorders exist. Certain disorders may be characterised by particular events, e.g. apneas, hypopneas, and hyperpneas.

Examples of respiratory disorders include Obstructive Sleep Apnea (OSA), Cheyne-Stokes Respiration (CSR), respiratory insufficiency, Obesity Hypoventilation Syndrome (OHS), Chronic Obstructive Pulmonary Disease (COPD), Neuromuscular Disease (NMD) and Chest wall disorders.

Chronic snoring is a condition affecting a considerable proportion of the population, estimated at 40% by some studies. During sleep, the patient's throat muscles relax, causing a narrowing of the pharynx. The consequence of this narrowing is an increase in the speed of the inhaled air caused by a venturi-type effect. The air excites the flexible part of the soft palate and uvula and these begin to vibrate noisily. The noise created in this way can reach up to 90 decibels.

Obstructive Sleep Apnea (OSA), a form of Sleep Disordered Breathing (SDB), is characterised by events including occlusion or obstruction of the upper air passage during sleep. It results from a combination of an abnormally small upper airway and the normal loss of muscle tone in the region of the tongue, soft palate and posterior oropharyngeal wall during sleep. The condition causes the affected patient to stop breathing for periods typically of 30 to 120 seconds in duration, sometimes 200 to 300 times per night. It often causes excessive daytime somnolence, and it may cause cardiovascular disease and brain damage. The syndrome is a common disorder, particularly in middle aged overweight males, although a person affected may have no awareness of the problem, e.g. see U.S. Pat. No. 4,944,310 (Sullivan).

A patient with respiratory insufficiency (a form of respiratory failure) may experience abnormal shortness of breath on exercise.

Obesity Hypoventilation Syndrome (OHS) is defined as the combination of severe obesity and awake chronic hypercapnia, in the absence of other known causes for hypoventilation. Symptoms include dyspnea, morning headache and excessive daytime sleepiness.

A range of therapies have been used to treat or ameliorate such conditions. Furthermore, otherwise healthy individuals may take advantage of such therapies to prevent respiratory disorders from arising. However, these can have a number of shortcomings.

### 1.2.2 Therapies

Various therapies, such as Continuous Positive Airway Pressure (CPAP) therapy, Non-invasive ventilation (NIV), Invasive ventilation (IV), and High Flow Therapy (HFT) have been used to treat one or more of the above respiratory disorders.

### 1.2.2.1 Respiratory Pressure Therapies

Respiratory pressure therapy is the application of a supply of air to an entrance to the airways at a controlled target pressure that is nominally positive with respect to atmosphere throughout the patient's breathing cycle (in contrast to negative pressure therapies such as the tank ventilator or cuirass).

Such respiratory therapies may be provided by a respiratory therapy system or device. Such systems and devices may also be used to screen, diagnose, or monitor a condition without treating it.

Continuous Positive Airway Pressure (CPAP) therapy has been used to treat Obstructive Sleep Apnea (OSA). The mechanism of action is hypothesized to be that continuous positive airway pressure acts as a pneumatic splint and may prevent upper airway occlusion, such as by pushing the soft palate and tongue forward and away from the posterior oropharyngeal wall. Treatment of OSA by CPAP therapy may be voluntary, and hence patients may elect not to comply with therapy if they find devices used, such as CPAP masks to provide such therapy one or more of: uncomfortable, difficult to use, expensive and aesthetically unappealing. If a mask is uncomfortable, or difficult to use a patient may not comply with therapy. Since it is often recommended that a patient regularly wash their CPAP mask, if a mask is difficult to clean (e.g., difficult to assemble or disassemble), patients may not clean their mask and this may impact on patient compliance. Additionally, some patients do not tolerate CPAP therapy well and so alternative therapies are available. some patients do not tolerate CPAP therapy well and so alternative therapies are available.

Another form of therapy system is a mandibular repositioning device.

### 1.2.2.2 Mandibular Repositioning

A mandibular repositioning device (MRD) or mandibular advancement device (MAD) is one of the treatment options for sleep apnea and snoring. It is an adjustable intra-oral appliance, available from a dentist or other supplier, which holds the lower jaw (mandible) in a forward position during sleep. The MAD is a removable device that a patient inserts into their mouth, prior to going to sleep, and removes, following sleep. Thus, the MAD is not designed to be worn all of the time. The MAD may be custom made, or produced in a standard form and include a bite impression portion designed to allow fitting to a patient's teeth. The mechanical protrusion of the lower jaw expands the space behind the tongue, puts tension on the pharyngeal walls to reduce collapse of the airway and diminish palate vibration.

In certain examples a mandibular advancement device may comprise an upper splint that is intended to engage with or fit over teeth on the upper jaw or maxilla and a lower splint that is intended to engage with or fit over teeth on the lower jaw or mandible. The upper and lower splints are connected together laterally via a pair of connecting rods. The pair of connecting rods are fixed symmetrically on the upper splint and on the lower splint.

In such a design the length of the connecting rods is selected such that when the MAD is placed in a patient's mouth the mandible is held in an advanced position. The length of the connecting rods may be adjusted to change the level of protrusion of the mandible. A dentist may determine a level of protrusion for the mandible that will determine the length of the connecting rods.

Some MADs are structured to push the mandible forward relative to the maxilla while other MADs, such as the ResMed Narval CC™ MAD are designed to retain the mandible in a forward position. This device also reduces or minimises dental and temporo-mandibular joint (TMJ) side effects. Thus, it is configured to minimises or prevent any movement of one or more of the teeth by the applied pressure. For instance, document US2005016547 discloses a MAD with an upper groove and a lower groove designed to align respectively with the upper jaw and the lower jaw. The grooves are linked together by two tie rods of such length that the lower jaw is maintained in an extended position relative to the upper jaw.

### 1.2.2.3 Bruxism Treatment

Bruxism is the excessive grinding of the teeth and/or excessive clenching of the jaw. Some treatment devices known as occlusal splints cover the teeth of the upper and/or lower jaw to mechanically protect them. There are available intra-oral devices including partial or full-coverage splints, i.e., splints fitting over some or all of the teeth. They are typically made of plastic (e.g., acrylic) and can be hard or soft. A lower appliance can be worn alone, or in combination with an upper appliance.

### 1.2.3 Screening, Diagnosis, and Monitoring Systems

Polysomnography (PSG) is a conventional system for diagnosis and monitoring of cardio-pulmonary disorders, and typically involves expert clinical staff to apply the system. PSG typically involves the placement of 15 to 20 contact sensors on a patient in order to record various bodily signals such as electroencephalography (EEG), electrocardiogra electrooculograpy (EOG), electromyography (EMG), etc. PSG for sleep disordered breathing has involved two nights of observation of a patient in a clinic, one night of pure diagnosis and a second night of titration of treatment parameters by a clinician. PSG is therefore expensive and inconvenient. In particular, it is unsuitable for home screening/diagnosis/monitoring of sleep disordered breathing.

Screening and diagnosis generally describe the identification of a condition from its signs and symptoms. Screening typically gives a true/false result indicating whether or not a patient's SDB is severe enough to warrant further investigation, while diagnosis may result in clinically actionable information. Screening and diagnosis tend to be one-off processes, whereas monitoring the progress of a condition can continue indefinitely. Some screening/diagnosis systems are suitable only for screening/diagnosis, whereas some may also be used for monitoring.

Clinical experts may be able to screen, diagnose, or monitor patients adequately based on visual observation of PSG signals. However, there are circumstances where a clinical expert may not be available, or a clinical expert may not be affordable. Different clinical experts may disagree on a patient's condition. In addition, a given clinical expert may apply a different standard at different times.

### 2 BRIEF SUMMARY OF THE TECHNOLOGY

The present disclosure is directed to a mandibular advancement device (MAD) used in the amelioration, treatment, or prevention of snoring or obstructive sleep apnea, by repositioning the lower jaw of a user in a forward position. The MAD may have either one or more of improved comfort, cost, efficacy, retention, ease of use and manufacturability, or at least providing a useful alternative to existing devices.

Disclosed is an intraoral device system comprising an intraoral device. The intraoral device can comprise an upper splint that can be structured to engage with at least a portion of one or more teeth on a maxilla of a patient. A lower splint can be structured to engage with at least a portion of one or more teeth on a mandible of the patient. A drive system can be provided for moving the lower splint with respect to the upper splint.

The intraoral device system can further comprise a feedback system for generating instructions for adjustment of the intraoral device (referred to herein as an adjusting feedback system or feedback system). The adjusting feedback system can comprise a memory storing machine-readable instructions. The adjusting feedback system can also comprise a control system including one or more processors configured to execute the machine-readable instructions. The machine-readable instructions can cause the control system to receive sensor data from one or more sensors associated with the patient. The machine-readable instructions can also cause the control system to process the sensor data to determine a respiratory event likelihood parameter of the patient. The machine-readable instructions can also cause the control system to output control instructions to the intraoral device that operate the drive system to move the lower splint with respect to the upper splint. This operation of the drive system can be responsive to the determined respiratory event likelihood parameter.

In some forms, the drive system may comprise a controller. In this form, the feedback system may be further configured to transmit the control instructions to the controller to operate the drive system.

In some forms, the drive system may be manually adjustable. In this form, the control system instructions may be outputted as an alert to the patient for manual operation of the drive system.

In some forms, the intraoral device system may further comprise one or more intraoral sensors to generate at least some of the sensor data. The one or more intraoral sensors may be associated with the intraoral device.

In some forms, the sensor data may comprise body position data. The body position data may indicate the position of the patient's body, e.g., back sleeping, side sleeping, etc. The processor may determine the respiratory event likelihood parameter by taking into account the body position data.

In some forms, the sensor data may comprise respiratory sound data indicating respiratory sounds of the patient. In this form, the processor may determine the respiratory event likelihood parameter by taking into account the respiratory sound data.

In some forms, the sensor data may comprise sleep cycle data. The sleep cycle data may indicate a sleep stage of the patient. In this form, the processor may determine the respiratory event likelihood parameter by taking into account the patients sleep stage.

In some forms, the control instructions may operate the drive system to automatically move the lower splint with respect to the upper splint. The lower splint may be moved with respect to the upper splint between an advanced position and a neutral position responsive to the determined respiratory event likelihood parameter.

In some forms, the determined respiratory event likelihood parameter may be correlated with a sleep apnea event. The correlation may be used as an indication of the patient's compliance.

In some forms, the system may further comprise an external computing device. The external computing device may be configured to communicate with the intraoral device for outputting control instructions. The control instructions may be for operation of the drive system to move the lower splint with respect to the upper splint responsive to the determined respiratory event likelihood parameter.

In some forms, the external computing device may comprise a user interface. The user interface may be configured to display the outputted alert. The user interface may allow the patient to interact with the manual operation of the drive system.

In some forms, the respiratory event likelihood parameter may have values comprising low, medium and high. The control instructions may be configured to operate the intraoral device in a different way depending on the determined value of the respiratory event likelihood parameter.

Also disclosed is an adjusting feedback system for an intraoral device. The adjusting feedback system can comprise an upper splint and a lower splint. The upper splint and the lower splint can be structured to engage with at least a portion of one or more teeth of a patient. The adjusting feedback system can have a drive system for moving the lower splint with respect to the upper splint.

The feedback system can comprise a memory storing machine-readable instructions and a control system. The control system can include one or more processors configured to execute the machine-readable instructions to implement a feedback process. The feedback process can facilitate adjustment of the intraoral device under operation of the drive system by: receiving sensor data from one or more sensors associated with the patient; processing the sensor data to determine a respiratory event likelihood parameter of the patient; and outputting control instructions for operation of the drive system to move the lower splint with respect to the upper splint responsive to the determined respiratory event likelihood parameter.

In some forms, the drive system may comprise a controller. In this form, the feedback system may be further configured to transmit the control instructions to the controller to operate the drive system.

In some forms, the drive system may be manually adjustable. In this form, the control system instructions may be outputted as an alert to the patient for manual operation of the drive system.

In some forms, the adjusting feedback system may further comprise one or more intraoral sensors to generate at least some of the sensor data. The one or more intraoral sensors may be associated with the intraoral device.

In some forms, the sensor data may comprise body position data. The body position data may indicate the position of the patient's body. In this form, the processor may determine the respiratory event likelihood parameter by taking into account the body position data.

In some forms, the sensor data may comprise respiratory sound data. The respiratory sound data may indicate respiratory sounds of the patient. In this form, the processor may determine the respiratory event likelihood parameter by taking into account the respiratory sound data.

In some forms, the sensor data may comprise sleep cycle data. The sleep cycle data may indicate a sleep stage of the patient. In this form, the processor may determine the respiratory event likelihood parameter by taking into account the patients sleep stage.

In some forms, the control instructions may operate the drive system. The control instructions may operate the drive system to automatically move the lower splint with respect to the upper splint. The splint may be moved between an advanced position and a neutral position responsive to the determined respiratory event likelihood parameter.

In some forms, the determined respiratory event likelihood parameter may be correlated with a sleep apnea event. The correlation may be used as an indication of the patient's compliance.

In some forms, an external computing device may be configured to communicate with the intraoral device for outputting control instructions. The control instructions may be for operation of the drive system to move the lower splint with respect to the upper splint responsive to the determined respiratory event likelihood parameter.

In some forms where the drive system is manually adjustable and the control system instructions are outputted as an alert to the patient for manual operation of the drive system, the external computing device may comprise a user interface. The user interface may be configured to display the outputted alert. In this form, the user interface may allow the patient to interact with the manual operation of the drive system.

In some forms, the respiratory event likelihood parameter may have values comprising low, medium and high. The control instructions may be configured to operate the intraoral device in a different way depending on the determined value of the respiratory event likelihood parameter.

Also disclosed is a method for controlling an operation in conjunction with an intraoral device. The intraoral device can comprise an upper splint and a lower splint structured to engage with at least a portion of one or more teeth of a patient. The operation can include, for example, generating an alarm to alert the patient as set forth previously, or for adjusting a position of the upper splint with respect to the lower splint.

The method can comprise receiving sensor data from one or more sensors associated with the patient. The method can also comprise processing the sensor data to determine a respiratory event likelihood parameter of the patient. The method can also comprise outputting control instructions for the operation. The outputting of the control instructions can be responsive to the respiratory event likelihood parameter.

In some forms, the control instructions may operate a drive system of the intraoral device to move the lower splint with respect to the upper splint responsive to the determined respiratory event likelihood parameter.

The intraoral device may be otherwise as set forth above.

### 3 BRIEF DESCRIPTION OF THE DRAWINGS

The present disclosure is illustrated by way of example, and not by way of limitation, in the figures of the accompanying drawings, in which like reference numerals refer to similar elements including:

### 3.1 Therapy

### 3.1.1 Respiratory System

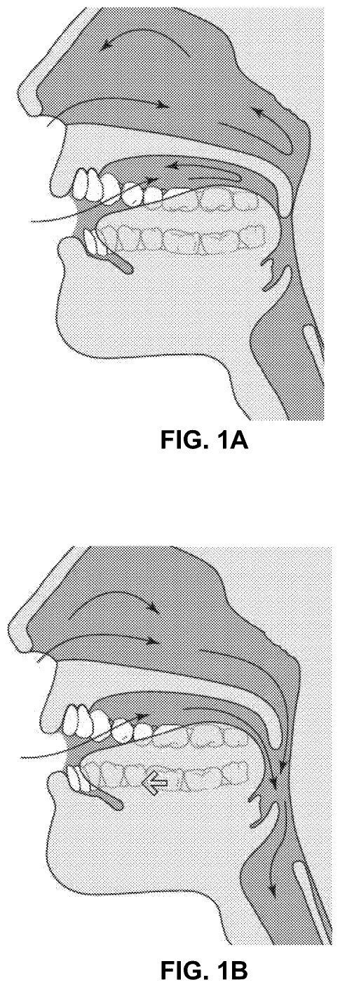

FIG. 1A shows a blocked airway due to the collapse of the muscles in the upper airway blocking the upper airway.

FIG. 1B shows how protrusion of the lower jaw expands the space behind the tongue to prevent or reduce blockage of the upper airway.

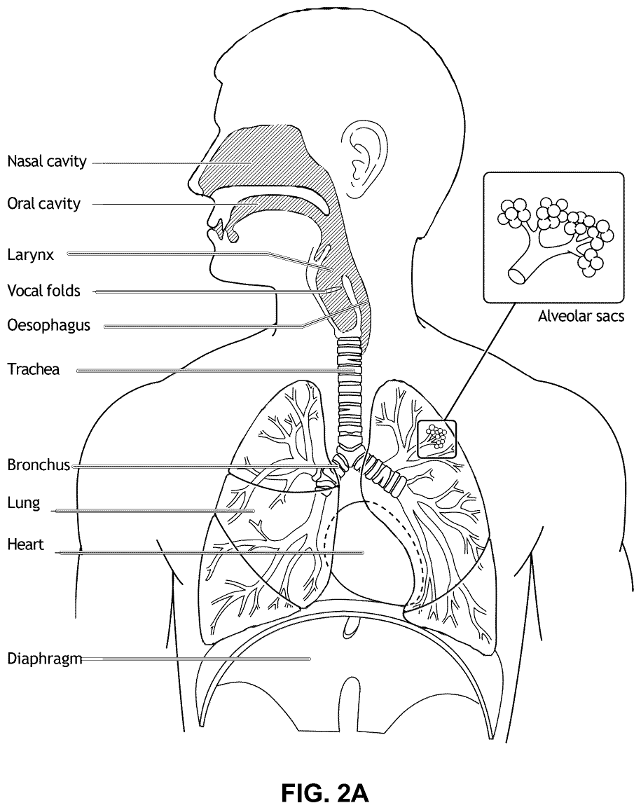

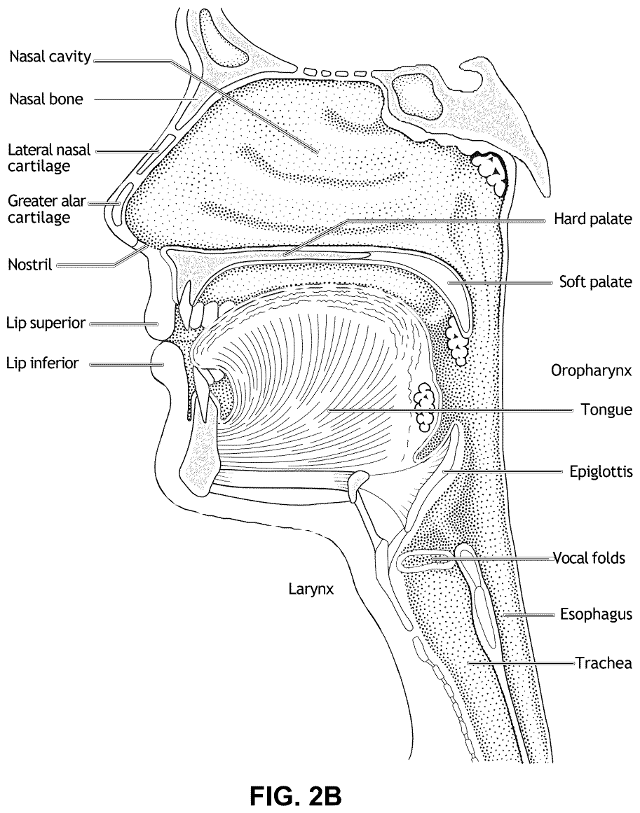

FIG. 2A shows an overview of a human respiratory system including the nasal and oral cavities, the larynx, vocal folds, oesophagus, trachea, bronchus, lung, alveolar sacs, heart and diaphragm.

FIG. 2B shows a view of a human upper airway including the nasal cavity, nasal bone, lateral nasal cartilage, greater alar cartilage, nostril, lip superior, lip inferior, larynx, hard palate, soft palate, oropharynx, tongue, epiglottis, vocal folds, oesophagus and trachea.

### 3.1.2 Mouth Anatomy

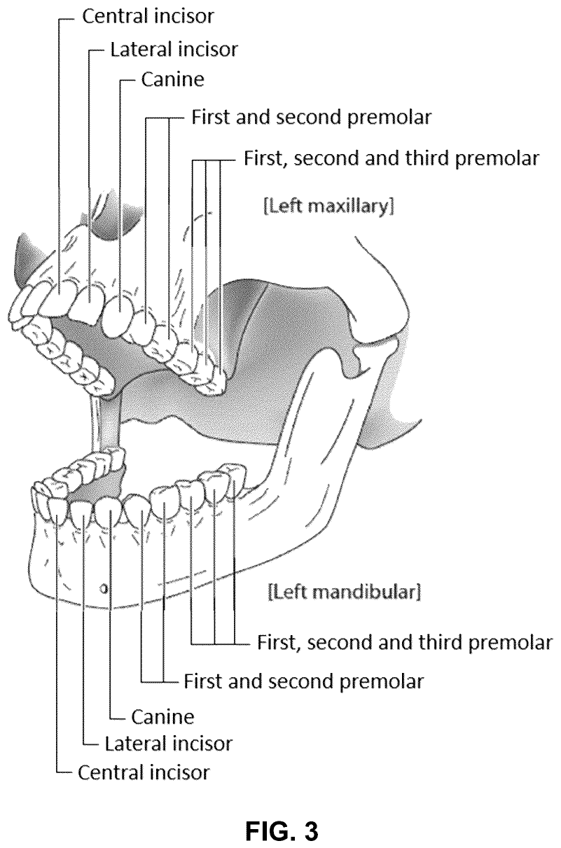

FIG. 3 shows an anterolateral view of an open human mouth showing the arrangement of the teeth on the maxilla and mandible jaws.

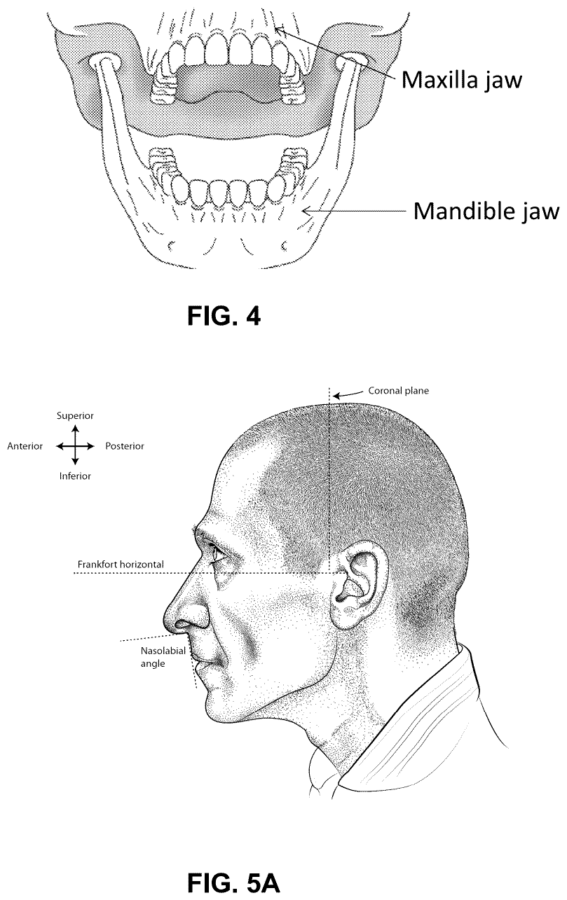

FIG. 4 shows a frontal (Antero-Posterior) view of an open human mouth showing the arrangement of the teeth on the maxilla and mandible jaws. Also indicated are the directions superior & inferior, and anterior & posterior.

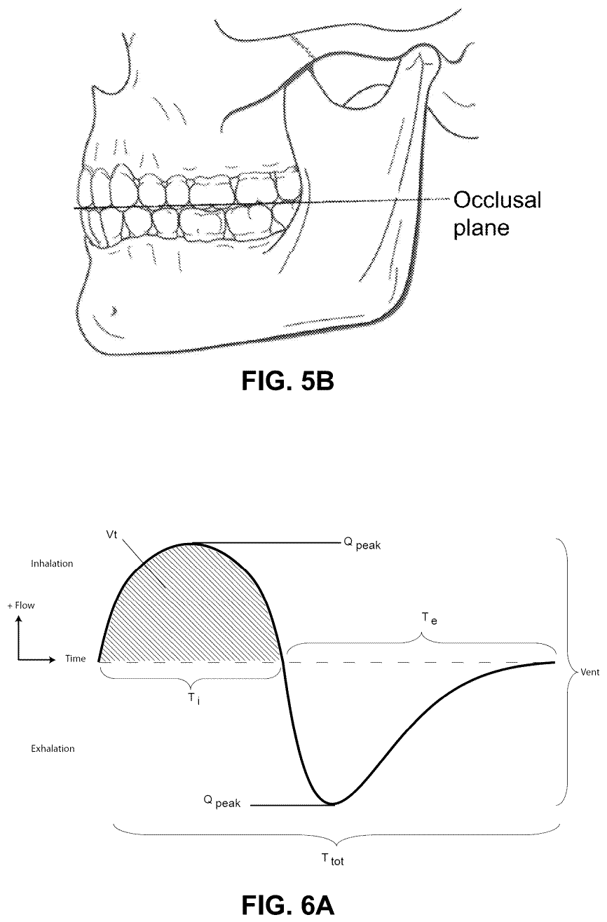

FIG. 5A shows a side view of a human face. The approximate locations of the Frankfort horizontal and nasolabial angle are indicated. The coronal plane is also indicated, as are the directions superior & inferior, and anterior & posterior.

FIG. 5B shows an anterolateral view of a closed human mouth with a line indicating the occlusal plane.

### 3.2 Breathing Waveforms

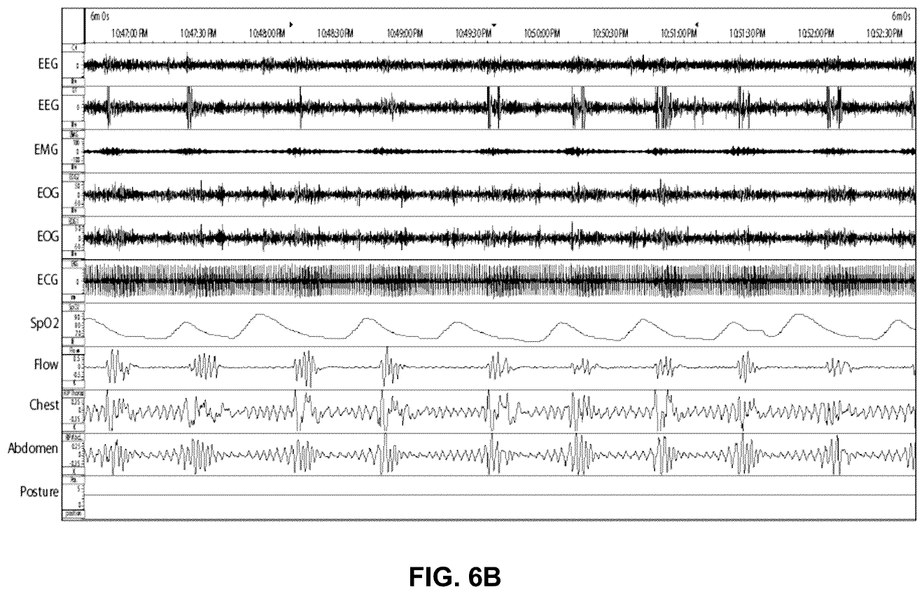

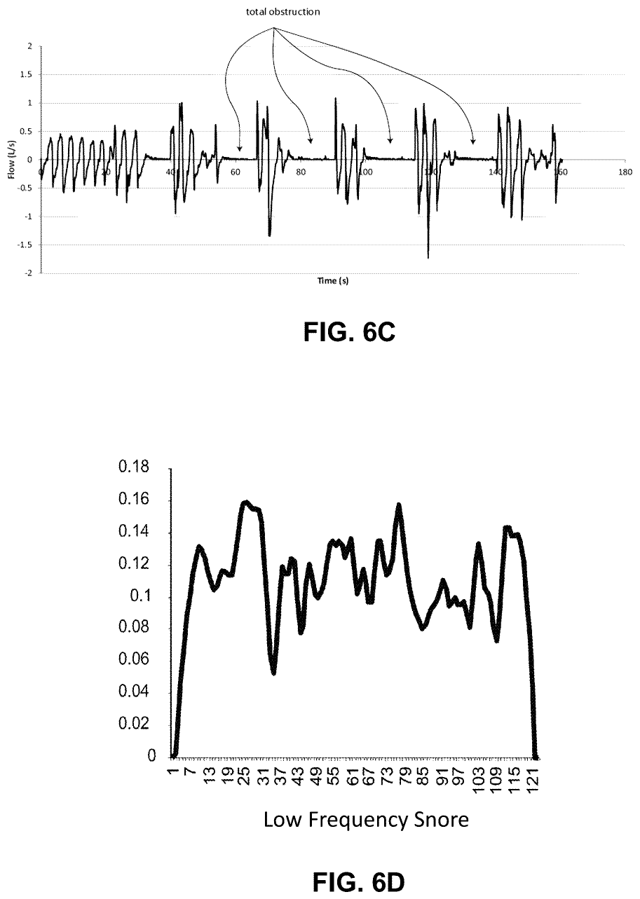

FIG. 6A shows a model typical breath waveform of a person while sleeping. The horizontal axis is time, and the vertical axis is respiratory flow rate. While the parameter values may vary, a typical breath may have the following approximate values: tidal volume Vt 0.5L, inhalation time Ti 1.6s, peak inspiratory flow rate Qpeak 0.4 L/s, exhalation time Te 2.4s, peak expiratory flow rate Qpeak-0.5 L/s. The total duration of the breath, Ttot, is about 4s. The person typically breathes at a rate of about 15 breaths per minute (BPM), with Ventilation Vent about 7.5 L/min. A typical duty cycle, the ratio of Ti to Ttot, is about 40%.

FIG. 6B shows polysomnography of a patient before treatment. There are eleven signal channels from top to bottom with a 6-minute horizontal span. The top two channels are both EEG (electoencephalogram) from different scalp locations. Periodic spikes in the second EEG represent cortical arousal and related activity. The third channel down is submental EMG (electromyogram). Increasing activity around the time of arousals represents genioglossus recruitment. The fourth & fifth channels are EOG (electro-oculogram). The sixth channel is an electocardiogram. The seventh channel shows pulse oximetry (SpO2) with repetitive desaturations to below 70% from about 90%. The eighth channel is respiratory flow rate using a nasal cannula connected to a differential pressure transducer. Repetitive apneas of 25 to 35 seconds alternate with 10 to 15 second bursts of recovery breathing coinciding with EEG arousal and increased EMG activity. The ninth channel shows movement of chest and the tenth shows movement of abdomen. The abdomen shows a crescendo of movement over the length of the apnea leading to the arousal. Both become untidy during the arousal due to gross body movement during recovery hyperpnea. The apneas are therefore obstructive, and the condition is severe. The lowest channel is posture, and in this example it does not show change.

FIG. 6C shows patient flow rate data where the patient is experiencing a series of total obstructive apneas. The duration of the recording is approximately 160 seconds. Flow rates range from about +1 L/s to about-1.5 L/s. Each apnea lasts approximately 10-15s.

FIG. 6D shows a scaled inspiratory portion of a breath where the patient is experiencing low frequency inspiratory snore.

### 3.3 Intraoral Device

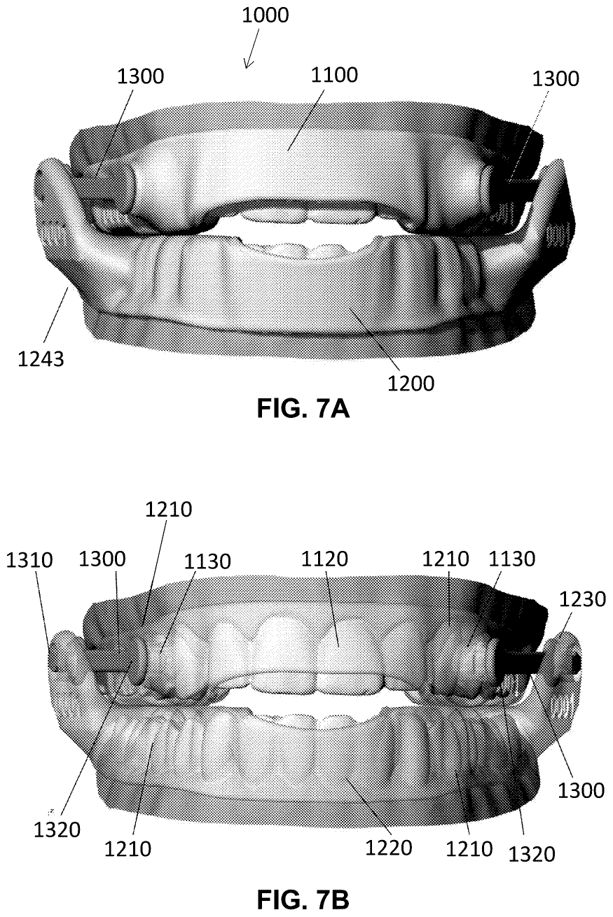

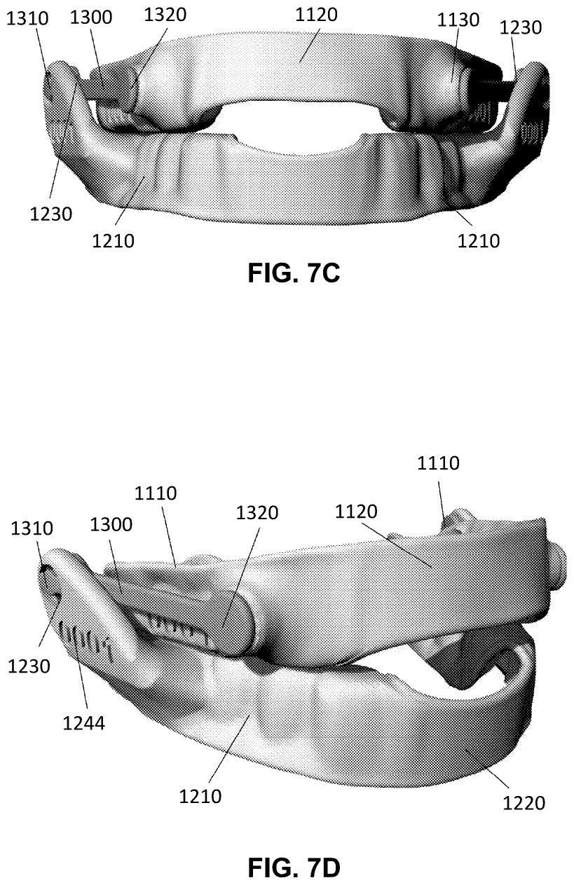

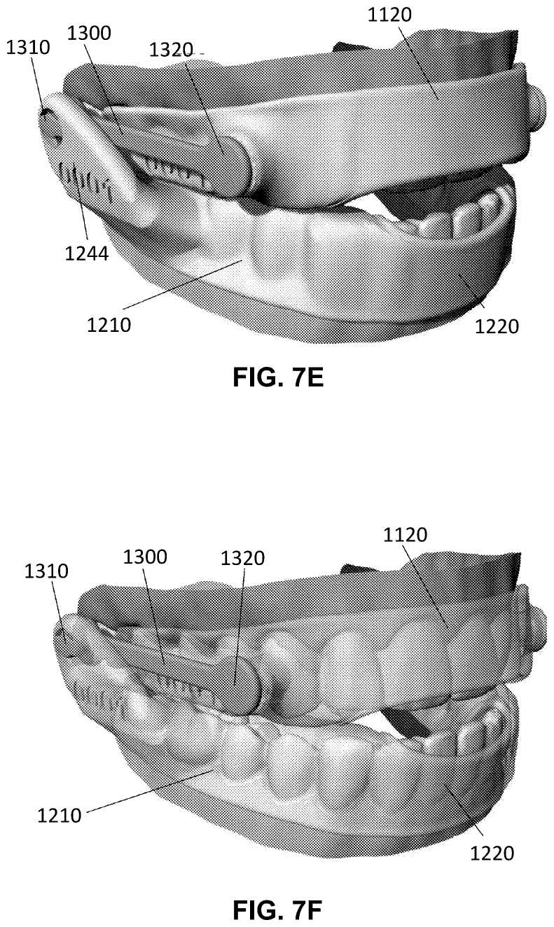

FIGS. 7A to 7F show an intra-oral device according to a first embodiment, in front views and perspective views.

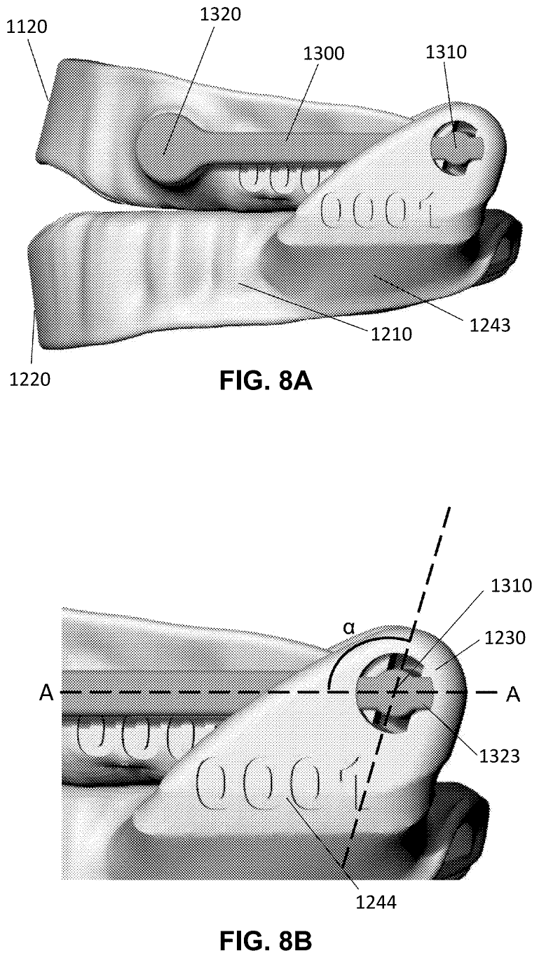

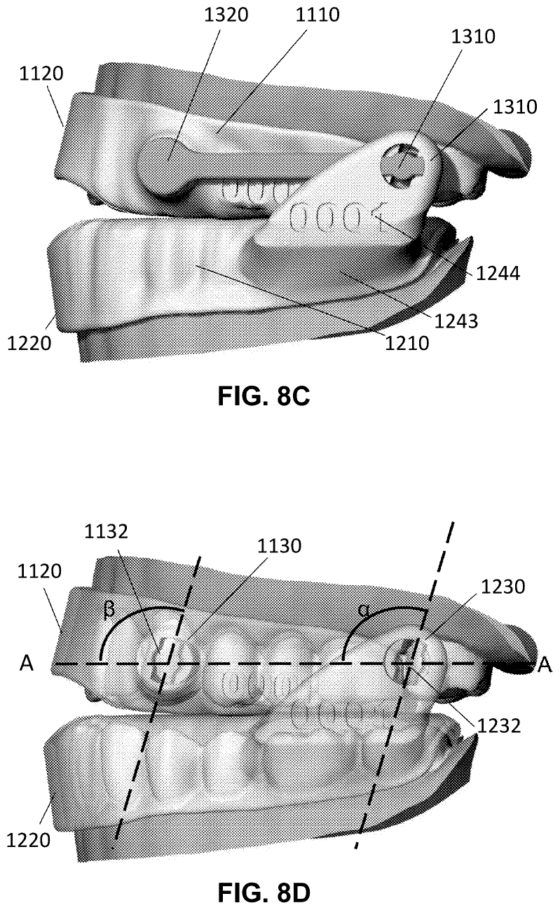

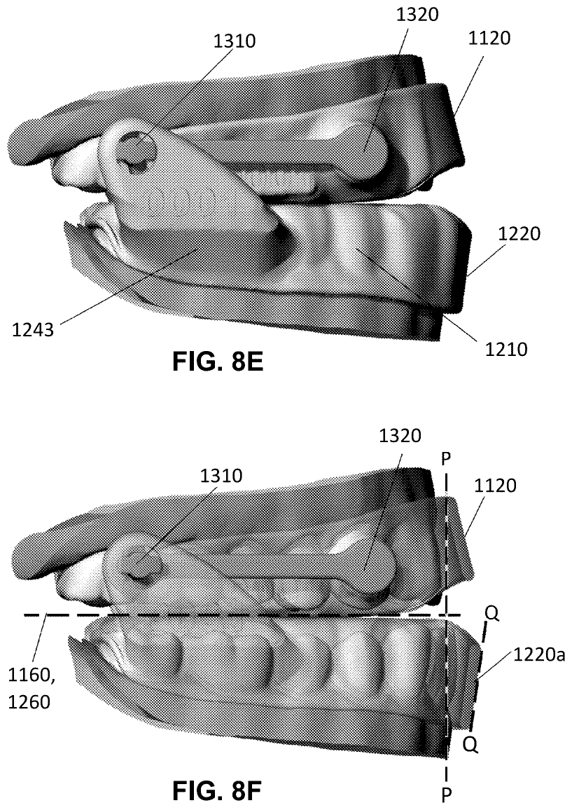

FIGS. 8A to 8F show side views of the upper and lower splints of a Mandibular Advancement Device (MAD).

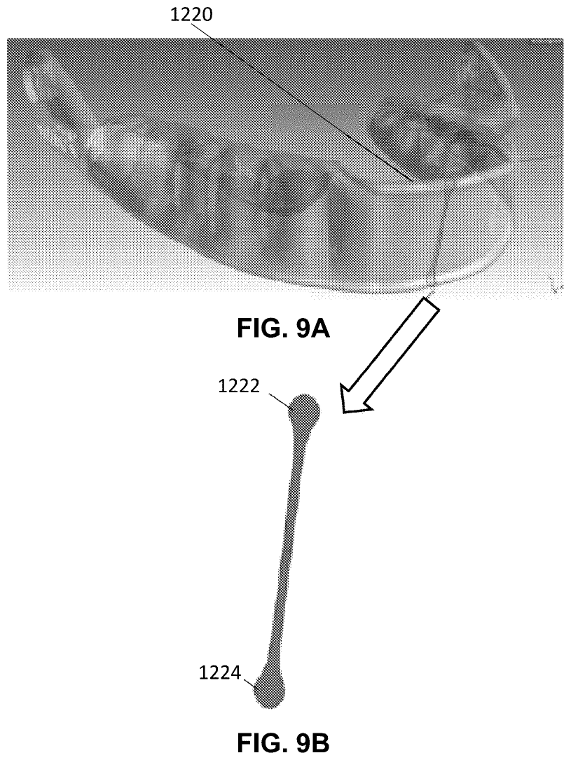

FIG. 9A shows an inclined mandible band portion of a lower splint according to the present technology.

FIG. 9B shows the profile of the mandible band portion respectively of an MAD according to the present technology.

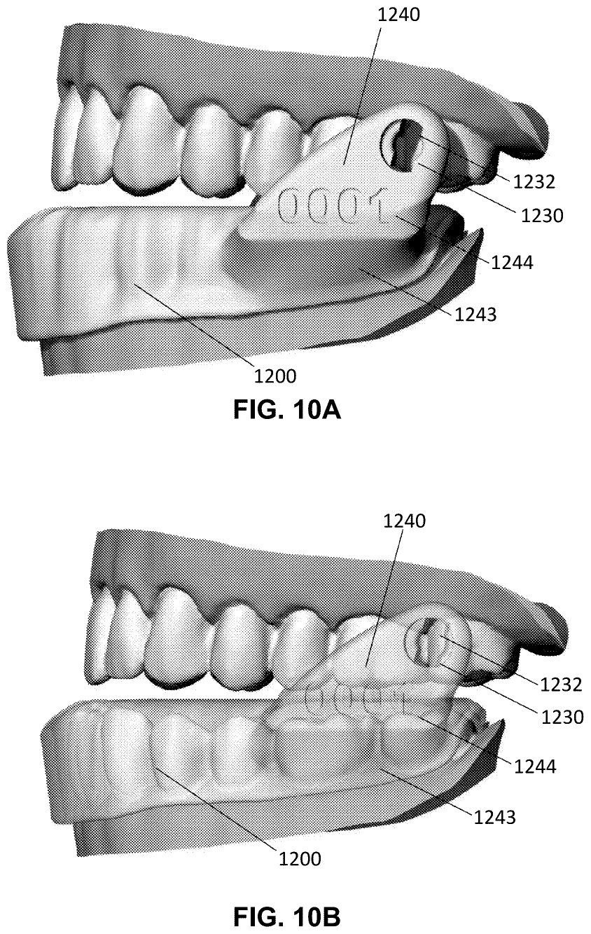

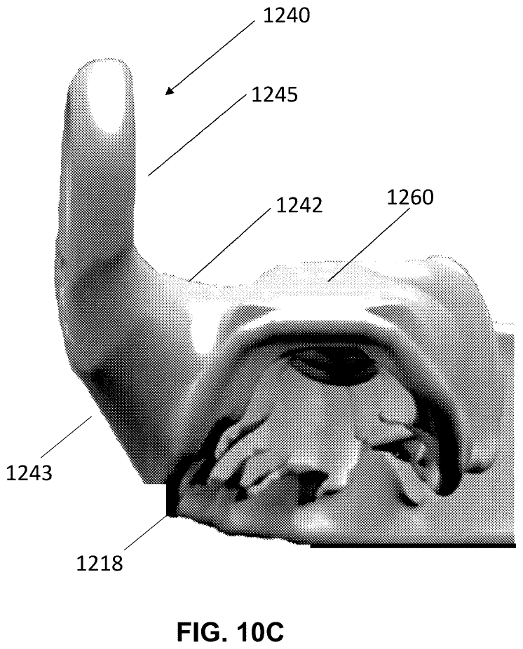

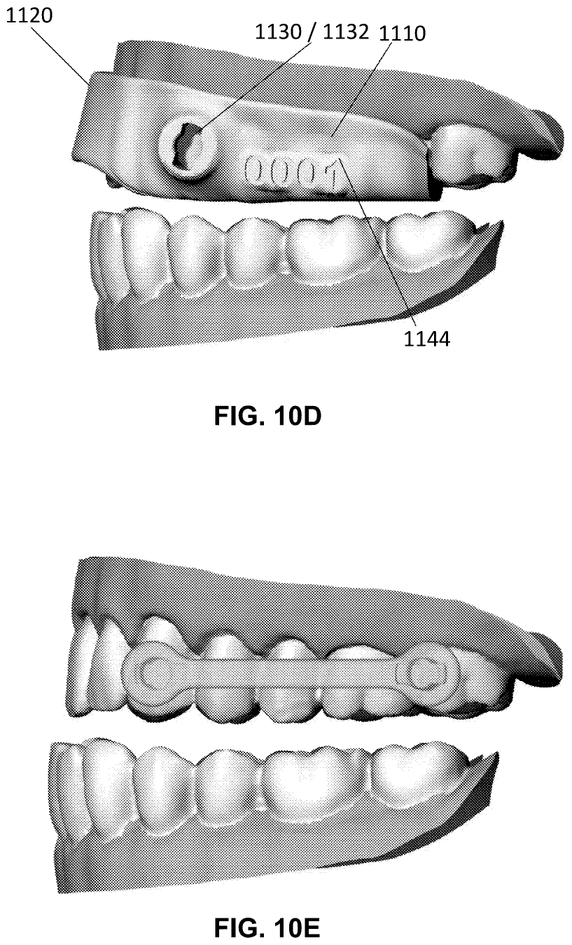

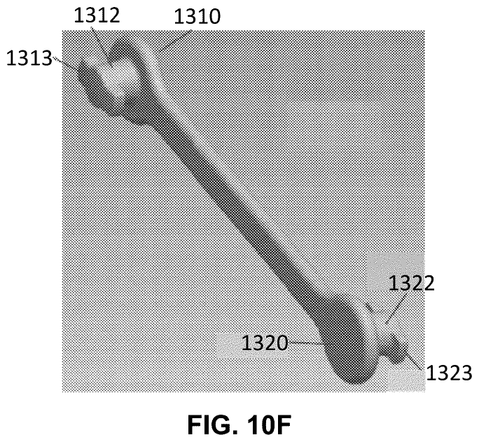

FIGS. 10A and 10B show side views of a lower splint of an MAD according to the present technology.

FIG. 10C shows a back view of a lower splint according to the present technology.

FIG. 10D shows a side view of an upper splint according to the present technology.

FIG. 10E shows a side view of a connecting rod according to the present technology.

FIG. 10F shows a perspective view of a connecting rod according to the present technology.

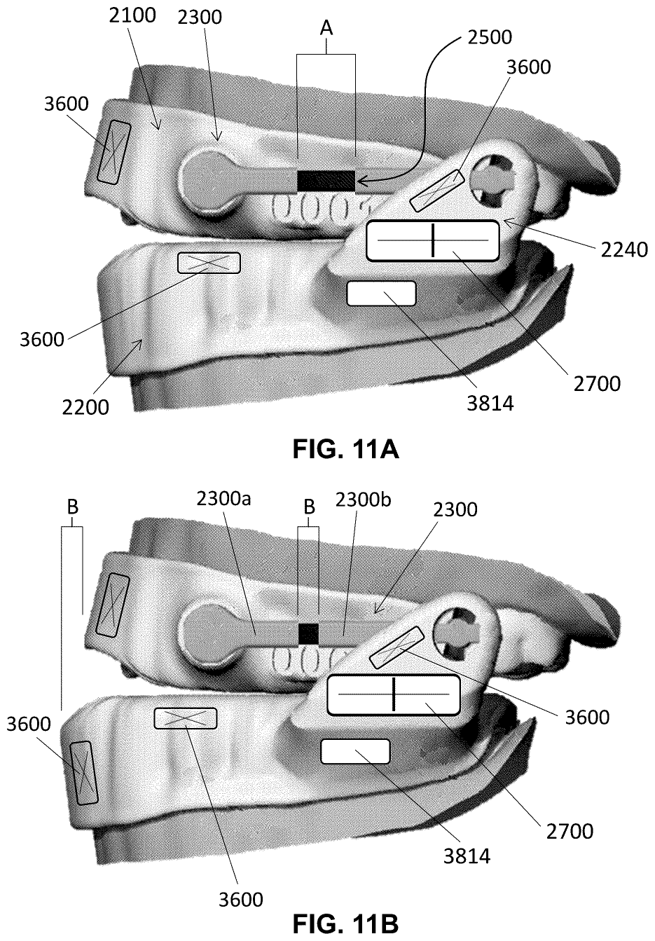

FIG. 11A shows a side view of the upper and lower splint of a Mandibular Advancement Device (MAD), where the lower splint is in a neutral position.

FIG. 11B shows a side view of the upper and lower splint of a Mandibular Advancement Device (MAD), where the lower splint is in an advanced position.

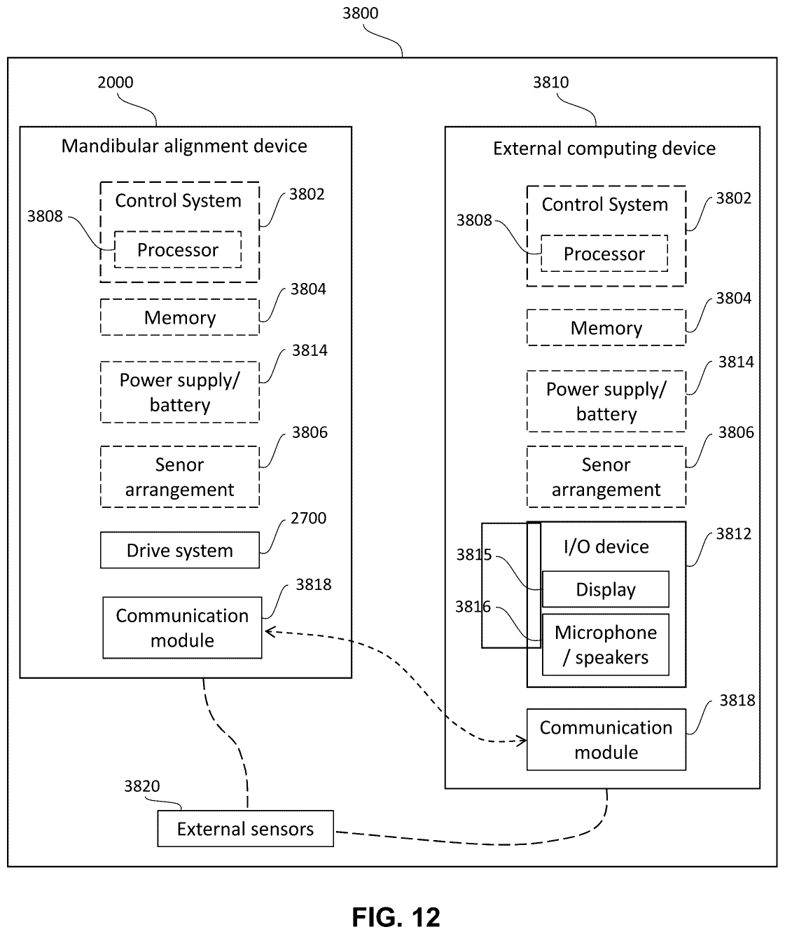

FIG. 12 is a block diagram of a feedback system according to some implementations of the present disclosure.

### 4 DETAILED DESCRIPTION OF EXAMPLES OF THE TECHNOLOGY

Before the present technology is described in further detail, it is to be understood that the technology is not limited to the particular examples described herein, which may vary. It is also to be understood that the terminology used in this disclosure is for the purpose of describing only the particular examples discussed herein, and is not intended to be limiting.

The following description is provided in relation to various examples which may share one or more common characteristics and/or features. It is to be understood that one or more features of any one example may be combinable with one or more features of another example or other examples. In addition, any single feature or combination of features in any of the examples may constitute a further example.

### 4.1 Embodiments of the Intraoral Device

Referring firstly to FIG. 7A, an intra-oral device or a mandibular advancement device (MAD) 1000 is shown fitted over a mould of an upper jaw and lower jaw including teeth. The intra-oral device or MAD comprises an upper splint 1100, a lower splint 1200 and a pair of connecting rods 1300 connecting the upper and lower splints 1100, 1200 together.

As seen in FIGS. 7A to 8F, the upper splint 1100 includes two maxilla or upper gutter portions 1110 designed or structured to fit over at least a portion of one or more teeth on each side of the maxilla or upper jaw. The upper gutter portions 1110 may cover a plurality of teeth in the region between the molars and canine on the maxilla. A maxilla or upper band portion 1120 is preferably provided between the two upper gutter portions 1110 to join the two upper gutter portions 1110 together. The upper band portion 1120 may be designed to extend between the two upper gutter portions 1110 across the front portion of the lateral and central incisors and may not engage with the internal or the external surface of these incisor teeth. Here, the upper band portion 1120 reduces the visual impact of the upper splint when inserted within the patient's mouth. Preferably, the upper splint 1100 is formed as a single piece with the upper gutter portions 1110 and the upper band portion 1120 integrally formed together.

However, it is noted that the upper splint 1100 may include a single upper gutter portion 1110 designed to fit over all of the teeth of the maxilla, thus no upper band portion 1120 would be required in such an upper splint. Such an upper splint may be more intrusive within the mouth. Such an upper splint may be used when the splint is used to treat Bruxism alone or simultaneously with treating obstructive sleep apnea.

The upper splint 1100 also may include one or more, but preferably a pair of upper splint connection points 1130, preferably one on each side of the upper splint 1100, to allow connection of a respective second rod end 1320 of each one of the pair of connecting rods 1300, to the upper splint 1100. As illustrated in FIGS. 7A and 8F, the upper splint connection points 1130 are preferably provided in the region of the canines. Preferably, the upper splint connection points 1130 are made as small as possible and may include a rounded shape to prevent irritation within the mouth. Preferably, the shape and size of the contact surface of the upper splint connection point 1130 substantially correspond to the shape and size of the second rod end 1320 (see FIGS. 7A to 7F and FIGS. 8A to 8F).

The lower splint 1200, as illustrated in FIGS. 7A to 8F, includes two mandible or lower gutter portions 1210 designed to fit over at least a portion of one or more teeth on each side of the mandible. The lower gutter portions 1210 may cover a plurality of teeth in the region between the molars and canine on the mandible. A mandible or lower band portion 1220 is preferably provided between the two lower gutter portions 1210 to join the two lower gutter portions 1210 together. The lower band portion 1220 may be designed to extend between the two lower gutter portions 1210 across the front portion of the lateral and central incisors and may not engage with the internal or the external surface of these incisor teeth. Here, the lower band portion 1220 reduces the visual impact of the lower splint 1200 when inserted within the patient's mouth. Preferably, the lower splint 1200 is formed as a single piece with the two lower gutter portions 1210 and the lower band portion 1220 integrally formed together.

However, it is noted that the lower splint 1200 may include a single lower gutter portion 1210 designed to fit over all of the teeth of the mandible respectively, thus no lower band portion 1220 is required in such a lower splint. Such a lower splint may be more intrusive within the mouth. Such a lower splint may be used when the splint is used to treat Bruxism alone or simultaneously with treating obstructive sleep apnea.

As shown in FIGS. 9A and 9B, the lower band portion 1220 may comprise rounded or smoothed edges, including at least one of the top and bottom edges to reduce or prevent irritation to the teeth or gums on the mandible or maxilla. In one arrangement, the top edge 1222 or bottom edge 1224 or both may have a tear-drop or drop-shape. FIGS. 9A and 9B depict the lower band portion. However, the upper band portion 1120 may also be configured in the same way as the lower band portion 1220 shown in FIGS. 9A and 9B, such that it may include rounded or smoothed top edges or bottom edges or both. Alternatively, or additionally, other outer edges of the upper or lower splint, particularly the edges of the gutter portion, may have rounded, smoothed or drop-shaped.

As particularly seen in FIG. 8F, the lower band portion 1220 may be inclined relative to a plane P-P perpendicular to the sliding plane surface 1160, 1260, which is parallel to the occlusal plane. The front surface 1220a of the lower band portion 1220 is inclined or angled (line Q-Q in FIG. 8F) to follow the angle of the incisors to prevent protrusion of the lower band portion 1220 into the inside of the lips. In other words, the front surface 1220a of the lower band portion 1220 angles slightly outwards from the bottom to the top in use. In a similar manner (as seen in FIG. 8F) the upper band portion 1120 may also be angled to follow the angle of the patients' incisors and may include rounded or smoothed top and bottom edges to reduce irritation of the maxilla gums. Such a design preferably prevents protruding too far inside of the lips.

Vestibular bands may be replaced by lingual bands and angles may be adjustable parameters. It is appreciated that the upper band portion 1120 or the lower band portion 1220 as such may be provided with different designs of MADs and the specific arrangement of other components such as the gutter portion design may vary.

As seen in FIGS. 7A to 8F the lower splint 1200 also may include one or more of lower splint connection points 1230, preferably a pair of points, one on each side of the lower splint 1200. Each lower splint connection point 1230 may be configured to allow connection to a first end 1310 of a respective one of the pair of connecting rods 1300 to the lower splint 1200 (FIGS. 7C, 7D and 8C). The lower splint connection points 1230 may be elevated relative to the lower gutter portions 1210. Preferably, the elevated lower splint connection points 1230 are adjacent the upper gutter portions 1110 on the upper splint 1100 (FIGS. 7D, 8A to 8D).

The lower splint connection point 1230 is preferably provided in the area of molars, such as the second molar (FIG. 10A). Thus, each one of the pair of connecting rods 1300 may be configured to substantially laterally connect the upper splint 1100 and the lower splint 1200, with the second end 1320 of the connecting rod 1300 connected to the upper splint 1100 and the first end 1310 of the connecting rod 1300 connected to the lower splint 1200 (FIGS. 7A-8F).

The lower splint connection points 1230 on the lower splint 1200 may be elevated in a position so that when the connecting rods 1300 are connected to the upper splint 1100 and lower splint 1200 the connecting rods 1300 are positioned substantially parallel with the Frankfort plane. In such an arrangement the traction force of the connecting rods 1300 is substantially parallel to the occlusal plane, which reduces the likelihood of the intra-oral device or MAD coming loose in use. This arrangement of the connecting rods 1300 is also advantageous for retaining the mandible in an advanced position.

As particularly illustrated in FIGS. 10A to 10C, each of the lower splint connection points 1230 may be formed in a wing structure 1240 that protrudes laterally from the lower splint 1200. The wing structure 1240 may have a curved structure to assist in supporting a patient's mucosa of the cheek when the intra-oral device or MAD is inserted in the patient's mouth. The wing structure 1240 may comprise a curved structure or filled portion 1243 connecting the substantially laterally extending wing base 1242 to the respective portion of the lower gutter portion (FIG. 10C). The filled portion 1243 is preferably contoured to provide support to the cheek so as to avoid dead space between the wing structure 1240/lower gutter portion 1220 and the mucosa of the cheek. The filled portion 1243 may extend from an outer side 1244 of the wing structure 1240 until the outer edge 1218 of the respective gutter portion in a substantially flat or even a slightly convex fashion, preferably without a concave portion.

Preferably, the wing structure 1240 is contoured to avoid edges or curvatures with small radii in order to avoid causing discomfort. Preferably, the length of the wing base 1242 and of the filled portion 1243 parallel to the direction of extension of the mandibular is selected so as to avoid edges, curvatures with small radii and/or dead space between the wing structure 1240/lower gutter portion and the mucosa of the check thereby increasing comfort. In other words, the wing structure may be less angular and the merging with the gutter may be optimized. The extremities of the wing structure may be slightly curved.

It is appreciated that the above wing structure and particularly the filled portion may also be provided with different designs of MADs and the specific arrangement of other components, such as the gutter or band portion design, may vary.

The wing structure may comprise an elevated portion 1245 elevating from the wing base 1242 (FIGS. 10A to 10C). Preferably, the lower splint connection point 1230 is located in the elevated portion 1245. The elevated portion 1245 may have a triangular configuration, although other shapes may be used. The lower splint connection point 1230 may be counter sunk within the wing structure 1240 to reduce irritation of a first attachment or rod pin 1312 of the connecting rod 1300 in a patient's mouth when the connecting rod 1300 is attached through the first slot 1232 (FIG. 10A) of the lower splint connection point 1230 of the lower splint 1200 and the MAD is in a patient's mouth (FIG. 7).

The thickness of the elevated portion 1245 can be adapted to accommodate a first rod pin 1312 so that the outer side or surface 1244 of wing structure 1240 is substantially planar. This means that preferably no portion or only a minor portion of the first rod pin 1312 protrudes from the outer side or surface 1244.

Referring now to FIG. 10F, the first and second rod pins 1312, 1322 may each comprise at least one pin protrusion 1313, 1323 extending laterally from the upper end of the rod pin. The lower splint connection point 1230 may comprise a recessed portion adapted to accommodate the at least one first pin protrusion 1313 (FIG. 10A). Preferably, the recessed portion is adapted to accommodate rotation of the at least one first pin protrusion 1313. The lower splint connection point 1230 may have a through hole with a shape adapted to the shape of the first attachment or rod pin 1312. The outer side 1244 may be arranged substantially parallel to an inner side. The inner side may be arranged as a substantially flat surface, preferably in an angle of (about) 90° to the wing base 1242 or the occlusal or sliding plane surface 1160, 1260 (FIG. 10C). This may improve the fixation of the connecting rod. The lower splint connection points 1230 of the lower splint 1200 may be formed as first slots 1232 configured to receive the first rod pins 1312 of the connecting rods 1300 (see FIG. 10A).

It is appreciated that the counter sunk connection point as well as the inner and outer surface may also be provided with different designs of MADs and the specific arrangement of other components, such as the particular wing structure arrangement in general, may vary.

The upper splint connection point(s) 1130 of the upper splint 1100 may be formed as second slot(s) 1132 configured to receive the second rod pin(s) 1322 of the connecting rod(s) (FIGS. 7A-8F, 10D, 10F). Preferably, the first slots 1232 and the second slots 1132 have complementary shapes to the shapes of the first rod pins 1312 and second rod pins 1322 respectively to facilitate insertion of the first rod pins 1312 and second rod pins 1322 into the first slots 1232 and the second slots 1132, respectively. The first slots 1232 and second slots 1132 are configured to allow the connecting rods 1300 to pivot or swivel within the slots 1132, 1232 in use to enable the opening and closing movement of the patient's mandible.

It is appreciated that the above described connection of the rod to the lower splint(s) may also be provided with different designs of MADs and the specific arrangement of other components, such as the gutter or band portion design, may vary.

The assembly and disassembly of the connecting rods 1300 to the upper splint 1100 and lower splint 1200 will now be described in relation to the lower splint using the first slot 1232, the first rod pin 1312 and the lower connection point 1230. However, it is to be understood that the same process is used to assemble and disassemble the upper splint and the connecting rods using the second slot 1132, the second rod pin 1322 and the upper connection point 1130. For assembly the first rod pins 1312 may be inserted into and through the first slot 1232 in the lower connecting point 1230 by aligning the first rod pins 1312 with the first slots 1232. Once inserted the connecting rod 1300 is rotated or pivoted around in the first slot 1232 to prevent the first rod pins 1312 from releasing out of the slot. For disassembly of the connecting rods 1300 from the first slot 1232 the connecting rod 1300 is preferably rotated or pivoted to realign the first rod pins 1312 with the first slots 1232 to allow removal of the rod pins through the slot.

To avoid detachment of the connecting rods 1300 during use or for detachment for cleaning the angle α, β of the first and second slot(s) 1132, 1232 are preferably set relative to the axis of the connecting rod 1300 to ensure that there is an at least one quarter turn in order to disassemble the connecting rod 1300. For instance, an at least one quarter turn in the clockwise direction is to be ensured in FIG. 8D. The longitudinal axis of the first slot 1232 and the longitudinal axis of the connecting rod 1300 in use (i.e., application by the user with closed mouth) may be arranged in a first obtuse angle α. Preferably the angle α is in a range of 90° to 170°, more preferably of 100° to 160 or of 110° or 130° to 150°, and most preferably of 105° to 135° (FIG. 8D).

Preferably, the longitudinal axis of the second slot 1132 and the longitudinal axis of the connecting rod 1300 in use (i.e., application by the user with closed mouth) are arranged in a second obtuse angle (β). Preferably, the second obtuse angle (β) is in a range of 90° to 170°, more preferably of 100° to 160°, or of 110° or 130° to 150° and most preferably of 105° to 135° (FIG. 8D). The angles α and β may have the same value. Moreover, angle α and/or β may preferably be 105°.

An angle of the longitudinal axis of the first slot 1232 to the sliding plane surface 1260 or occlusal plane may be adjustable. Similarly, the angle of the longitudinal axis of the second slot 1132 to the sliding plane surface 1160 may also be adjustable. The angle may be adjusted considering the relative orientation of the connecting rod 1300 to the sliding plane surface 1160, 1260 or occlusal plane. With such a tailored design parameter, the risk of an unintended disassembly can be further reduced.

It is appreciated that the above described angular relationship between the longitudinal axis of the slots to the longitudinal axis of the connecting rod in use may also be provided with different designs of MADs and the specific arrangement of other components, such as the gutter or band portion design or the general wing design, may vary.

The degree of advancement of the mandible may be defined by the length of the connecting rods 1300. In some forms, the connecting rods may be static, i.e., rods 1300 may be formed in a range of predetermined lengths. For example, the rods may be formed in lengths from 20 mm to 40 mm, such as 21 mm to 36 mm, with incrementing sizes for example of 0.5 mm, 1 mm or 1.5 mm. In this form, the connecting rods 1300 may be manually attachable to and detachable from the splints 1100, 1200 as set forth previously, so as to allow interchanging of the connecting rods 1300 for different lengths to adjust the level of mandible advancement.

Referring now to an embodiment of the MAD 2000 as shown in FIGS. 11A and 11B. Alternatively, the length of the connecting rods 1300 may be adjustable, i.e., as adjustable connecting rods 2300, to facilitate adjustments in the level of mandible advancement. In some forms, the adjustable connecting rods may be configured as tie rods comprising two elements 2300a, 2300b.

The elements 2300a, 2300b may be adjusted relative to each other by a mechanism 2500. As set forth below, the mechanism may take various forms and hence, is represented in FIGS. 11A and 11B as a block element 2500.

In some forms, the mechanism may include a first of the two elements 2300a being a threaded rod and the other of the two elements 2300b being a rod having a threaded bore intended to receive the threaded rod. By screwing or unscrewing one of the elements relative to the other, the length of the tie rod may be adjusted from a ‘resting’ distance ‘A’ to a distance ‘B’. That is, a relative position of the tie rods may be adjusted to change the level of mandible advancement by a distance ‘B’ as shown in FIG. 11B.

In a variation, the tie rods 2300a, 2300b may comprise two threaded bores into which is screwed a rod whose ends have an inverse pitch (i.e., thread). The rod may be turned in order to lengthen or shorten the tie rods between positions ‘A’ and ‘B’.

In a further variation, the tie rods 2300a, 2300b may be configured such that each comprises a cylinder in which a rod slides. The free end of the rod is equipped with a piston which delimits two chambers. The ties rods of this variation may be configured such that hydraulic pressure can be exerted on the piston to displace the rod of each of the tie rods 2300a, 2300b and hence lengthen or shorten the tie rods.

As set forth above, it is to be understood that the connecting rods 2300 can have a variety of configurations and sizes different to those set forth above, so long as the above-described control of the mandibular position between ‘A’ and ‘B’ is accomplished. For example, the connecting rods may not be located laterally, i.e., on opposing sides of the upper and lower splints. Rather, a single connecting rod may be provided for adjusting the level of mandible advancement. Such a rod may be arranged on a single side of the upper and lower splints, or alternatively, the rod may be located centrally of the upper and low splints.

In some forms, the MAD 2000 may not comprise connecting rods, i.e., tie rods, rather, the upper and lower splints 2100, 2200 may be adjustable relative to each other by other means. For example, a geared arrangement may be provided between the two splints, such that a drive gear may be configured to move the splints relative to each other.

In some forms, mechanisms 2500 set forth above, may be manually adjusted to change the level of mandibular advancement. In other forms, as set forth in more detail below, the mechanism 2500 may be automatically adjustable in response to events that effect the patient's respiration, i.e., respiratory events.

In some forms, the mechanism 2500 set forth above may from part of a drive system 2700 configured for controlling the automatic adjustment of the level of mandibular advancement. In the forms of the mechanism set forth above, the drive system 2700 may include a motor configured to control an operation of the mechanism 2500. For example, the motor may be configured to rotate by screwing and unscrewing, the threaded rods for adjusting the level of mandibular advancement. In other forms, e.g., where the tie rods are adjusted by an application of hydraulic pressure, the drive system may alternatively comprise one or more pneumatic or hydraulic actuators configured to exert pressure on the pistons so as to displace the rods.

An operation of the drive system 2700, e.g., the actuator, motor, etc., can be controlled manually, e.g., by a patient operating the drive system. In some forms, the drive system may not be controlled manually, rather, a position of the lower splint may be manually controlled by the patient e.g., screwing and unscrewing the rod elements as set forth above. In this case, the patient can override control of the position of the lower splint to adjust its position ‘by hand’.

In other forms, as set forth in more detail below, the drive system 2700 may be automatically operated to achieve a desired anatomical position of the patient's mandible.

Referring to FIG. 12, the drive system 2700 can be controlled by a feedback system 3800 for advancing a position of the lower splint 2200, i.e., to advance a patient's mandible. The feedback system can include a control system 3802, a memory device 3804 and a sensor arrangement 3806 comprising one or more sensors 3600 configured for acquiring sensor data, e.g. measuring physiological data of a patient, for monitoring events that may affect the patient's respiration. As set forth in more detail below, said events are monitored to determine a likelihood of the patient experiencing a sleep apnea event.

The control system 3802 can include one or more processors 3808 (hereinafter, processor 3808). The control system 3802 is generally used to control (e.g., actuate) the drive system 2700 in response to the events that affect, i.e., impair, the patient's respiration. The control system 3802 is also generally used to analyse the sensor data obtained and/or generated by the one or more sensors 3600 of the system 3800 to determine the likelihood of the patient experiencing a sleep apnea event.

The processors of the control system can be configured to process the sensor data to determine a likelihood parameter, i.e., a ‘probability’, of the patient experiencing the sleep apnea event based on the acquired sensor data acquired relating to a monitored event. For example, a monitored even may be a patient's sleeping position/orientation. In this example, acquired sensor data that indicates the patient is sleeping on their front may be allocated a ‘low’ likelihood parameter, i.e., a side-sleeping patient has a low probability of experiencing a sleep apnea event.

The processor 3808 can be a general or special purpose processor or microprocessor. While one processor 3808 is shown in FIGS. 12, the control system 3802 can include any suitable number of processors (e.g., one processor, two processors, five processors, ten processors, etc.) that can be in a single housing, or located remotely from each other.

The memory device 3804 stores machine-readable instructions that are executable by the processor 3808 of the control system. In particular, the memory device 3804 may comprise a plurality of processor control instructions executable by the processors 3808 to implement a feedback process that facilitates the adjustment of the intraoral device, i.e., of the lower splint, under operation of the drive system 2700.

As set forth in more detail later, in some forms of the control instructions, the operation of the drive system may occur automatically, i.e., being responsive to respiratory events. In other forms, the control instructions may be configured to alert a patient as to adjustments they can manually apply to the lower splint, i.e., being applied after a respiratory event has occurred.

The memory device 3804 can be any suitable computer readable storage device or media, such as, for example, a random or serial access memory device, a hard drive, a solid state drive, a flash memory device, etc. While one memory device 3804 is shown in FIGS. 12, the feedback system 3800 can include any suitable number of memory devices (e.g., one memory device, two memory devices, five memory devices, ten memory devices, etc.). The memory device(s) can be coupled to and/or collocated with the control system.

In some forms, one or more components of the feedback system 3800 may be coupled to and/or positioned (or in some cases, embedded/integrated) within, the MAD itself. For example, while not shown, the control system 3802 may be integrated as a ‘controller’ being contained within the MAD. In addition, the MAD may comprise the drive system 2700, the memory device 3804 and the one or more sensors 3600 as part of either, or both of, the upper and lower splint. That is, said components can be arranged with respect to the upper or lower splint as ‘onboard’ components of the feedback system 3800.

In this form, the MAD may be a self-contained and self-operated system for adjusting mandibular advancement. Such a MAD may be used by a patient without connection to other devices.

In some forms, the feedback system 3800 may include an external computing device 3810 (e.g., a smart phone, a laptop, etc.). The external computing device may comprise an I/O device 3812 (i.e., a user interface) configured for receiving information from, and outputting information to, the patient during in-use.

The one or more components of the feedback system 3800, as set forth above, may be coupled to, and/or be part of, the external computing device 3810. For example, and as indicated in FIG. 12 in dotted-outline, the control system 3802 may be an existing control system of the external computing device 3810 configured with one or more processors 3808, memory 3804 and a power supply 3814 to perform the processing and detections (from the received sensing signals of the sensors). In this regard, the feedback system 3800 may be split, i.e., dividing across both the MAD 2000 and the external computing device 3810. For example, and as set forth in more detail later, the MAD 2000 may comprise the one or more sensors 3600, while the external computing device 3810 may comprise the control system 3802.

As set forth above, in some forms, the MAD and the computing device may both comprise memory devices 3804, control systems 3802 having processors 3808, etc., (as set forth above) for cooperatively operating advancement of the MAD lower splint 2200. For example, the ‘onboard’ components of the MAD may be configured to apply initial processing such as to filter noise, perform an initial calibration of the sensors, apply thresholds to sensing data to restrict data range, etc.

In some forms, the external computing device 3810, e.g., smart phone, may be configured to run an application or a program adapted to process the data received from the sensors.

The computing device 3810 in the form of e.g., the smart phone can also comprise a user/display interface 3815 configured as part of the I/O device 3812 for displaying information relating to operation of the feedback system 3800. The computing device 3810 may further comprise a separate input interface such as a microphone/speaker 3816 or a character (e.g., alpha-numeric) input module, or both. As set forth in more detail later, the user/display interface can be configured to allow the user to manually control operation of the MAD, e.g., to remotely control the e.g., motor to adjust the lower splint 2200 with respect to the upper splint 2100.

The memory device 3804 of the computing device 3810 (and/or the MAD) may comprise a plurality of processor control instructions for controlling the processors 3808. As set forth in more detail later, the memory device 3804 may also be configured to store data gathered by the sensors of the MAD for use as e.g., a ‘patient profile’.

The computing device 3810 may also comprise a communication module 3818 for receiving and sending the sensing signals between the one or more sensors 3600 of the MAD 2000 and the computing device 3810. For example, the one or more sensors of the MAD may be operably coupled with the processing system of the computing device by a wireless link such as Bluetooth, Wi-Fi or other communications interface.

As best shown in FIGS. 11A and 11B, the one or more sensors 3600 may be incorporated, e.g., embedded, into either or both of the upper or lower splints 2100, 2200. The sensor(s) 3600 may be located at an anterior of the splints, at sides of the splints, or a combination of both. In some forms, the one or more sensors may be incorporated into only the lower or upper splint. In further forms, the one or more sensors may be specifically incorporated into the wing structure 2240 of the lower splint.

The MAD 2000 may also comprise a communication module 3818, e.g., comprising a transponder, for wireless transmission of the sensing signals to the communications module 3818 of the computing device 3810. In this regard, the transponder may be integrated together with the sensor(s) in either or both of the upper and lower splints.

The MAD 2000 can also comprise an integrated power supply 3814, e.g., a battery, for providing power to the one or more sensors 3600, communication module 3818 and the drive system 2700 (e.g., motor, actuators, etc.).

The control system 3802 is generally used to control (e.g., actuate) the drive system 2700 in response to the events that affect, i.e., impair, the patient's respiration.

The one or more sensors of the MAD may include motion sensors, positional sensors, heart rate sensors, temperature sensors, peripheral oxygen saturation (SpO2) sensor, photoplethysmogram (PPG) sensors, electrooculography (EOG) sensors, electromyography (EMGs) sensors, analyte sensors, infrared (IR) sensors, sound sensors, or any combination thereof.

For example, an optical sensor using red, infrared, and/or green, could be used to calculate a photoplethysmogram. Subsequently, parameters such as pulse rate (PR), pulse rate variability (PRV) and SpO2 can be determined. If respective sensors are placed on a periphery of the patient, e.g., in contact with their gums, the peripheral arterial tone may also be measured.

Further, while the motion sensor is noted above in broad terms, the motion sensor may be specifically one or more inertial sensors, such as accelerometers, gyroscopes, and magnetometers. These types of motion sensors may be selected, i.e., utilised according to their optimal use-case.

In some forms of the motion sensors, the motion sensors may be configured to detect motion or acceleration associated with arterial pulses, such as pulses in or around the gums of the patient and in particular, those proximal to the sensor locations, e.g., proximal to the wing structure 1240. The motion sensors in this form may be configured to detect features of the pulse shape, speed, amplitude, or volume that may be analysed to indicate characteristics of the patient's respiration, i.e., respiratory events such as sleep apnea.

In some forms, the external computing device 3810 can also comprise one or more sensors configured for use with the feedback system 3800. The sensors of the computing device can be existing sensors of the computing device, e.g., inertial sensors, light sensors, etc. The sensor data detected and measured by the sensors of the computing device may be utilised together with the sensor data detected and measured by the MAD.

Additionally, or alternatively, the feedback system 3800 may include one or more external sensors 3820 being external to the sensors 2600 of the MAD and computing device. The external sensors 3820 may be configured as part of e.g., a home ‘IoT’ hub, or other device arranged with respect to the patient. The external sensors 3820 may be configured to detect and measure sensor data related the patient, or alternatively, properties of the patient's surrounding environment that may relate to a likelihood of the patient experiencing e.g., sleep apnea. The sensor data detected and measured by the external sensors may be utilised together with the sensor data detected and measured by the MAD 2000 and computing device 3810.

As set forth above, the sensors 3600 may be configured together with the control system 3802 of the computing device 3810 to implement actuation of the drive system 2700, e.g., the motor, actuator, etc., based on the data measured by the one or more sensors. As set forth below, the one or more sensors may be utilised in various ways to determine a likelihood parameter of a patient experiencing a sleep apnea event.

In some forms, motion sensors in the form of one or more accelerometers can be configured to measure a patient's sleeping position. The data relating to the patient's sleeping position, i.e., their body position being on a side, back or front, can be used to indicate whether the patients' airways are occluded and hence, a likelihood of the patient experiencing a sleep apnea event.

For example, a patient suffering from positional sleep apnea is less likely to experience a sleep apnea when sleeping on their side or front than when sleeping on their back. Accordingly, the sensors 3600, 3820 can be configured to measure and record sensor data relating to the sleeping position of the patient, for then determining a likelihood parameter of the patient having a sleep apnea event. For example, a patient moving from a side sleeping orientation to a back sleeping orientation may experience a narrowing of their airways as their soft palate moves to at least partially occlude their airways when in the back sleeping orientation. In this example, the patient moving to the back sleeping orientation may be allocated a ‘high’ likelihood parameter. That is, there is a high probability of the patient experiencing a sleep apnea event.

In other examples, a patient who is sleeping on their front may be allocated a ‘low’ likelihood parameter; and a patient who is sleeping on their side may be allocated a ‘medium’ likelihood parameter. It is noted that a ‘low’, ‘medium’ or ‘high’ quantification of the likelihood parameter is only an example, and other quantifications are possible, e.g., percentages, etc.

Detection of a patient's body position (or reposition) may be used to indicate an occlusion (or a risk of an occlusion) and hence whether the patient may experience a sleep apnea event. Accordingly, the likelihood parameter can be used to determine an adjustment of the lower splint by e.g., the motor, actuator, etc., to open the patient's airways in response to the event. For example, a ‘low’ likelihood parameter may not require the lower splint to be advanced; a ‘medium’ likelihood parameter may result in the lower splint being partially advanced; and a ‘high’ likelihood parameter may result in the lower splint being fully advanced.

The feedback system may also allow the lower splint to move away from the advanced position towards a neutral position when the probability of a sleep apnea event occurring reduces, e.g., the likelihood parameter changes from ‘high’ to ‘medium’. For example, a patient rolling from their front to a side sleeping orientation may result in the control system generating a ‘medium’ likelihood parameter and in turn, actuating the drive system to increase a protrusion of the patient's mandible to open their airways. If the patient moves from the side sleeping orientation to the front sleeping position again, the likelihood parameter may change to ‘low’, resulting in the control system actuating the drive system to move the lower splint towards the neutral position.

Moving the lower splint from the advanced position when the patient's airways are not occluded can position their mandible in a more natural, resting position. Advantageously, this can increase, i.e., maximise the patient comfort during use of the MAD.

The magnitude by which the lower splint is adjusted based on the likelihood parameter may be determined by processing various types of data collected by the sensor(s). For example, the sensors may be configured to detect a flow of air moving past the MAD, such that an occlusion of the airway may be quantified. Based on the measurement of airway occlusion, together with e.g., positional information of the patient, a position of the lower splint may be determined. Advantageously, utilising more than one source of data may allow the patient's mandible to be advanced by only a required amount to return sufficient airflow for the patient's breathing. In effect, the patient's mandible is only adjusted by a necessary amount, i.e., without applying excessive advancement of the lower splint so as to create unnecessary strain and discomfort for the patient's jaw.

In some forms, the one or more sensors may be configured to measure and detect when a patient wearing the MAD is experiencing a sleep apnea event. Upon detection of such an event, the control system can be configured to operate the drive system, e.g., motor, actuator, etc., to advance the patient's mandible for opening their airway. In this case, the lower splint may be fully advanced to maximally open the patient's airways. In this case, i.e., the occurrence of a sleep apnea, the likelihood parameter for the patient experiencing a sleep apnea event would be allocated the highest possible value, e.g., if quantified with percentages, the likelihood parameter would be considered ‘100%’.

Conversely, when the one or more sensors detect a completion of the sleep apnea event, the lower splint may be returned from the advanced position into a neutral position, i.e., a position whereby the patient's mandible is in a more natural, resting position. As set forth above, the control system can be configured to optimise a position of the patient's mandible, i.e., magnitude of advancement, based on the occurrence of a sleep apnea event, such that the mandible is only advanced when needed, and by an appropriate magnitude.

In some forms, the one or more sensors and control system can be configured to monitor an occurrence, frequency, etc., of a patient's sleep apnea events to determine the patient's adherence, i.e., compliance to MAD therapy. In this form, the feedback system may be configured together with e.g., a smart phone, to display the patient's adherence to therapy for e.g., tracking, monitoring, etc., their use of MAD therapy.

In some forms, the MAD may additionally or alternatively be operatively coupled with a system and method for analysing sleep-related parameters as described in PCT publication WO2021214640, the contents of which are incorporated herein in their entirety. The system and method for analysing sleep-related parameters may utilise one or more sensors and can be configured for tracking a patient's sleep apnea events to determine the patient's adherence to therapy.

Monitoring the occurrence of sleep apnea events, as set forth above, may also be utilised as an indication of efficacy of the MAD therapy. That is, a reduction in the occurrence of sleep apnea events may indicate that use of the MAD has improved the patient's sleep. In effect, the control system 3802 may allocate a ‘low’ respiratory event likelihood parameter.

In some forms of the feedback system, an adjustment of the lower splint may be effected by said reduction in sleep apnea events. For example, in response to the detection of said ‘low’ likelihood parameter, i.e., effective MAD therapy, the control system 3802 may be configured to reduce a frequency, magnitude, etc., of mandible advancement. That is, the magnitude, frequency, etc., of the lower splint advancement may be modified based on the likelihood parameter.

In effect, the MAD can be configured to increase patient comfort as a result of the patient's improved sleep. Advantageously, this may promote continued use of the MAD, i.e., a more comfortable mandible position may incentivise a patient to continue their MAD therapy.

Additionally, or alternatively, the feedback system may not be configured to adjust a position of the lower splint based on said reduction in sleep apnea events. Rather, the determination of e.g., a ‘low’ likelihood parameter, may be communicated to the patient as feedback on their compliance to therapy. For example, such communication may be displayed on the user/display interface 3815 of the computing device 3810. This information may be used by the patient, or by their health care professional for assessing their use of the MAD.

In some forms, the feedback system may be configured to monitor patient adherence to MAD therapy by utilising the one or more sensors of the MAD in combination with a case, i.e., housing, protective cover, etc., for the MAD device. The one or more sensors of the MAD may be configured to detect when the MAD is place within, or removed from, the case as an indication of use of the MAD. For example, removal of the MAD device from the case may indicate the patient is about to wear the MAD. Conversely, detection of the MAD being returned, i.e., placed into the case may indicate the patient has concluded their use of the MAD.

As set forth previously, a patient's compliance with MAD therapy may be an indication of a reduced likelihood of a respiratory event such as sleep apnea. This determination may be used in several ways: to modify advancement of the lower splint; and/or inform a patient of their compliance via the user/display interface 3815 of computing device 3810.

In a variation, the case provided for tracking a patient's adherence to MAD therapy (as set forth above) may be a case also configured for cleaning the MAD between uses. Advantageously, providing the patient with a case that can clean their MAD may encourage the patient to store their MAD therein between uses, such that the patient's use of the MAD device, i.e., adherence, is improved.

In some forms, the one or more sensors of the MAD can be configured to measure and detect respiratory sounds produced by a patient during sleep. The sensors may be configured to detect particular characteristics of the sounds that, once processed by the control system, can be used to determine the likelihood parameter of a respiratory event. The detected sounds may be e.g., snoring, wheezing, etc. and can be used to determine whether the MAD is configured correctly in the patient's mouth, i.e., whether the patient's mandible has been suitably advanced for opening the patient's airways.

The control system can be configured to allocate a likelihood parameter based on the sounds detected. For example, a loud snoring sound may indicate a ‘high’ likelihood the patient is experiencing a sleep apnea. Based on the ‘high’ likelihood parameter, the control system may activate the drive system, e.g., motor, actuator, etc., of the MAD to adjust the position of the patient's mandible. Advancement of the MAD based on e.g., snoring sounds, can be automatically adjusted to alleviate the respiratory event upon detection of the sleep sounds. As set forth previously, the MAD may be configured to stop advancement of the lower splint once the snoring has ceased. In this way, the feedback system may optimise the opening of the patient's airways, such that the airway is sufficiently opened to alleviate the respiratory event, without applying excessive strain on the patient's mandible.

In some forms, the one or more sensors may be configured to detect when the patient enters a sleep cycle, i.e., REM, for then activating the drive system, e.g., motor, actuator, etc., of the MAD to advance the patient's mandible. In this form, the feedback system may be configured to allocate a ‘high’ likelihood parameter once the patient enters the REM stage to activate advancement of the MAD at that stage.

Advancing the patient's mandible only after they enter REM sleep can provide more comfort in early stages of their sleep where the patient may be more sensitive to discomfort caused by advancement of their mandible. That is, the MAD therapy is only applied when it is required, to maximise patient comfort. Advantageously, this may provide the patient with a more relaxing, natural beginning to their sleep, i.e., assisting the patient to fall asleep, prior to advancement of their mandible.

In the form set forth above, i.e., whereby the MAD is configured to detect REM sleep, the one or more sensors may include electromyogram (EMG) sensors for detecting electrical activity from a muscle. The EMG sensors may be configured to measure decreases in muscular activity as indications that the patient is in REM sleep, or may soon enter REM sleep. Further, the one or more sensors may include flow sensors configured to detect surges (i.e., sudden accelerations or decelerations) in the patient's breathing rate as an indication of REM sleep.

Additionally, or alternatively, the feedback system as set forth above may be configured to adjust the lower splint position during the patient's REM sleep stage. That is, although the lower splint may be advanced during the patient's REM sleep stage, the control system may apply minor adjustments to the lower splint position to optimise the patient's breathing and comfort. For example, if a sleep apnea event is detected, the lower splint may be further adjusted to further open the patient's airways. Conversely, when the sleep apnea event has ceased, the lower splint may be slightly adjusted back towards the neutral position.

In some forms, the control system may be configured to cyclically activate advancement of the mandible to minimise a duration of the patient's mandible being in the advanced position. That is, and as best shown in FIG. 11B, the MAD may be activated to move the mandible by a distance ‘B’ for opening the patient's airways only for short periods of time during a sleep session, rather than for a long period of time, e.g., for an entire night. The short periods of mandible advancement may place less strain, i.e., fatigue on the patient's anatomy, i.e., jaw muscles, etc., resulting in greater comfort of the patient.

In some forms, the short periods of advancement may be spaced by relatively longer periods where the mandible, i.e., lower splint, is not advanced, or is advanced by a lesser extent. In other forms, the duration of mandible advancement may be chosen by the patient by e.g., interaction with a smart phone connected with the MAD. In further forms, the duration of mandible advancement may be determined by the one or more sensors' detection of a likelihood of respiratory events as set forth previously.

In some forms, the control system of the MAD may be configured to activate the drive system for mandible advancement at the time of insertion into the patient's mouth. That is, when the one or more sensors detect that the patient is wearing the MAD, the drive system can begin to advance the mandible immediately. In this form, the MAD may be configured such that the patient's mandible is not repeatedly adjusted during a sleeping period. That is, the mandible will only be moved into the advanced position once, before returning from the advanced position (i.e., into a natural, resting position) near an end of the sleep period, i.e., just before the patient wakes.

Additionally, the MAD can be configured to adjust the mandible position during a sleep period. That is, the MAD may advance the mandible position immediately, and also adjust the mandible position during the sleep period as set forth previously.

In either form, the advancement may begin slowly such that the patient can acclimatise to the strain applied to their lower jaw. In some case, the mandibular advancement may be slow enough that the patient may fall asleep before the lower splint has reached its optimal advancement. Advantageously, this can minimise patient discomfort whilst wearing the MAD.

In some forms, the one or more sensors and the control system may be configured to detect events during a patient's sleep that affect their respiration, then present the patient with feedback, e.g., recommendations, relating to changes that may be made to the mandibular advancement. The recommendations may be provided to the patient following a period of sleep, such that the patient can manually adjust the mandibular advancement according to the recommendation, prior to a following period of sleep, i.e., the following night.

The recommendations set forth above may be presented to the patient as an alert, i.e., a communication, via the user interface. In this form, the drive system may be operated by the patient responding to the alert. That is, the lower splint will only be adjusted in position once the patient actions the recommendations of the alert.

The alerts or recommendations may be based on a likelihood parameter as set forth previously. For example, if the patient is determined as having a ‘medium’ likelihood parameter, the display may suggest a corresponding advancement of the lower splint be applied.

In variations, information in addition to the recommended mandibular advancement may be provided. For example, an indication of SDB events may be provided to the patient, in addition to duration of sleep, interruptions that may affect sleep quality, etc.

While the method of providing patient feedback (set forth above) indicates a manual adjustment of the lower splint by the patient, the MAD may be configured to automatically adjust in response to the recommended mandibular advancement. In this form, the MAD may be additionally configured to adjust the advancement of the lower splint once the MAD is removed from the patient's mouth, i.e., after a period of sleep. In effect, the MAD can be adjusted in readiness for use by the patient before the next period of sleep. Advantageously, this can introduce minor adjustments to the mandibular advancement in between sleep sessions, such that the patient is less able to perceive the further advancement of their mandible.

As set forth previously, the storage of the computing device may be configured to store data gathered by the sensors. Additionally, the storage may be configured to store an analysis of the sensor data and records of MAD therapy applied to the patient, consistent with that set forth above, i.e., adjustments in the magnitude, duration, etc., of mandible advancement in response to the detection of respiratory events. In some forms, the data stored in the memory may be used to generate a profile associated with the patient, i.e., a patient profile. The patient profile may be configured to be presented to the patient on the user interface of the computing device to provide the patient with feedback on their MAD therapy.

In some forms, the patient profile may be integrated with the application or program of the computing device. The application, being adapted to process the data received from the sensors, may also be configured to allow the patient to interact with their MAD therapy. In some forms, the application may be configured to allow the patient to control operation of the MAD within set therapeutic limits for their MAD therapy. For example, the patient may be able to reduce a magnitude of advancement if they have too much discomfort with the existing advancement of their mandible. It is anticipated that other parameters of the patient's MAD therapy may be adjusted, as set forth previously, e.g., duration of mandible advancement, etc.

In some forms, the application may be configured to enable the patient or another user to manually input, i.e., self-report, information that cannot be determined from the sensors of the MAD. For example, the patient may be able to input demographic information associated with the patient, phenotype information associated with the patient, biometric information associated with the patient, medical information associated with the patient, self-reported patient feedback, sleep parameters associated with the patient (e.g., sleep-related parameters recorded from one or more earlier sleep sessions).

The demographic information can include, for example, information indicative of an age of the patient, a gender of the patient, a race of the patient, a family history of insomnia or sleep apnea, or any combination thereof.

The phenotype information associated with the patient can include, for example, height, weight, skin colour, hair colour or eye colour, body composition, blood pressure, and information regarding other observable physical or biochemical traits of the patient.

The medical information can include, for example, information indicative of one or more medical conditions associated with the patient, medication usage by the patient, or both. The medical information data can further include a multiple sleep latency test (MSLT) result or score and/or a Pittsburgh Sleep Quality Index (PSQI) score or value.

The self-reported user feedback can include information indicative of a self-reported subjective sleep score (e.g., poor, average, excellent), a self-reported subjective stress level of the patient, a self-reported subjective fatigue level of the patient, a self-reported subjective health status of the patient, a recent life event experienced by the patient, or any combination thereof. One or more of the pieces of information set forth above may be taken into account when analysing the sensor data and applying MAD therapy to the patient.

### 4.2 Glossary

For the purposes of the present technology disclosure, in certain forms of the present technology, one or more of the following definitions may apply. In other forms of the present technology, alternative definitions may apply.

### 4.2.1 General

4.2.2 Respiratory cycle

Apnea: According to some definitions, an apnea is said to have occurred when flow falls below a predetermined threshold for a duration, e.g. 10 seconds. An obstructive apnea will be said to have occurred when, despite patient effort, some obstruction of the airway does not allow air to flow. A central apnea will be said to have occurred when an apnea is detected that is due to a reduction in breathing effort, or the absence of breathing effort, despite the airway being patent. A mixed apnea occurs when a reduction or absence of breathing effort coincides with an obstructed airway.

Expiratory portion of a breathing cycle: The period from the start of expiratory flow to the start of inspiratory flow.

Flow limitation: Flow limitation will be taken to be the state of affairs in a patient's respiration where an increase in effort by the patient does not give rise to a corresponding increase in flow. Where flow limitation occurs during an inspiratory portion of the breathing cycle it may be described as inspiratory flow limitation. Where flow limitation occurs during an expiratory portion of the breathing cycle it may be described as expiratory flow limitation.

Hypopnea: According to some definitions, a hypopnea is taken to be a reduction in flow, but not a cessation of flow. In one form, a hypopnea may be said to have occurred when there is a reduction in flow below a threshold rate for a duration. A central hypopnea will be said to have occurred when a hypopnea is detected that is due to a reduction in breathing effort. In one form in adults, either of the following may be regarded as being hypopneas:
(i) a 30% reduction in patient breathing for at least 10 seconds plus an associated 4% desaturation; or
(ii) a reduction in patient breathing (but less than 50%) for at least 10 seconds, with an associated desaturation of at least 3% or an arousal.

Hyperpnea: An increase in flow to a level higher than normal.

Inspiratory portion of a breathing cycle: The period from the start of inspiratory flow to the start of expiratory flow will be taken to be the inspiratory portion of a breathing cycle.

Patency (airway): The degree of the airway being open, or the extent to which the airway is open. A patent airway is open. Airway patency may be quantified, for example with a value of one (1) being patent, and a value of zero (0), being closed (obstructed).

### 4.2.3 Anatomy

4.2.3.1 Anatomy of the skull

Frontal bone: The frontal bone includes a large vertical portion, the squama frontalis, corresponding to the region known as the forehead.

Mandible: The mandible forms the lower jaw. The mental protuberance is the bony protuberance of the jaw that forms the chin.

Maxilla: The maxilla forms the upper jaw and is located above the mandible and below the orbits. The frontal process of the maxilla projects upwards by the side of the nose, and forms part of its lateral boundary.

Nasal bones: The nasal bones are two small oblong bones, varying in size and form in different individuals; they are placed side by side at the middle and upper part of the face, and form, by their junction, the “bridge” of the nose.

Nasion: The intersection of the frontal bone and the two nasal bones, a depressed area directly between the eyes and superior to the bridge of the nose.

Occipital bone: The occipital bone is situated at the back and lower part of the cranium. It includes an oval aperture, the foramen magnum, through which the cranial cavity communicates with the vertebral canal. The curved plate behind the foramen magnum is the squama occipitalis.

Orbit: The bony cavity in the skull to contain the eyeball.

Parietal bones: The parietal bones are the bones that, when joined together, form the roof and sides of the cranium.

Temporal bones: The temporal bones are situated on the bases and sides of the skull, and support that part of the face known as the temple.

Zygomatic bones: The face includes two zygomatic bones, located in the upper and lateral parts of the face and forming the prominence of the cheek.

### 4.2.3.2 Anatomy of the Respiratory System

Diaphragm: A sheet of muscle that extends across the bottom of the rib cage. The diaphragm separates the thoracic cavity, containing the heart, lungs and ribs, from the abdominal cavity. As the diaphragm contracts the volume of the thoracic cavity increases and air is drawn into the lungs.

Larynx: The larynx, or voice box houses the vocal folds and connects the inferior part of the pharynx (hypopharynx) with the trachea.

Lungs: The organs of respiration in humans. The conducting zone of the lungs contains the trachea, the bronchi, the bronchioles, and the terminal bronchioles. The respiratory zone contains the respiratory bronchioles, the alveolar ducts, and the alveoli.

Nasal cavity: The nasal cavity (or nasal fossa) is a large air filled space above and behind the nose in the middle of the face. The nasal cavity is divided in two by a vertical fin called the nasal septum. On the sides of the nasal cavity are three horizontal outgrowths called nasal conchae (singular “concha”) or turbinates. To the front of the nasal cavity is the nose, while the back blends, via the choanae, into the nasopharynx.

Pharynx: The part of the throat situated immediately inferior to (below) the nasal cavity, and superior to the oesophagus and larynx. The pharynx is conventionally divided into three sections: the nasopharynx (epipharynx) (the nasal part of the pharynx), the oropharynx (mesopharynx) (the oral part of the pharynx), and the laryngopharynx (hypopharynx).

### 4.3 Other Remarks

A portion of the disclosure of this patent document contains material which is subject to copyright protection. The copyright owner has no objection to the facsimile reproduction by anyone of the patent document or the patent disclosure, as it appears in Patent Office patent files or records, but otherwise reserves all copyright rights whatsoever.

Unless the context clearly dictates otherwise and where a range of values is provided, it is understood that each intervening value, to the tenth of the unit of the lower limit, between the upper and lower limit of that range, and any other stated or intervening value in that stated range is encompassed within the technology. The upper and lower limits of these intervening ranges, which may be independently included in the intervening ranges, are also encompassed within the technology, subject to any specifically excluded limit in the stated range. Where the stated range includes one or both of the limits, ranges excluding either or both of those included limits are also included in the technology.

Furthermore, where a value or values are stated herein as being implemented as part of the technology, it is understood that such values may be approximated, unless otherwise stated, and such values may be utilized to any suitable significant digit to the extent that a practical technical implementation may permit or require it.

Furthermore, “approximately”, “substantially”, “about”, or any similar term used herein means +/−5-10% of the recited value.

Unless defined otherwise, all technical and scientific terms used herein have the same meaning as commonly understood by one of ordinary skill in the art to which this technology belongs. Although any methods and materials similar or equivalent to those described herein can also be used in the practice or testing of the present technology, a limited number of the exemplary methods and materials are described herein.

When a particular material is identified as being used to construct a component, obvious alternative materials with similar properties may be used as a substitute. Furthermore, unless specified to the contrary, any and all components herein described are understood to be capable of being manufactured and, as such, may be manufactured together or separately.

It must be noted that as used herein and in the appended claims, the singular forms “a”, “an”, and “the” include their plural equivalents, unless the context clearly dictates otherwise.

All publications mentioned herein are incorporated herein by reference in their entirety to disclose and describe the methods and/or materials which are the subject of those publications. The publications discussed herein are provided solely for their disclosure prior to the filing date of the present application. Nothing herein is to be construed as an admission that the present technology is not entitled to antedate such publication by virtue of prior invention. Further, the dates of publication provided may be different from the actual publication dates, which may need to be independently confirmed.

The terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

The subject headings used in the detailed description are included only for the ease of reference of the reader and should not be used to limit the subject matter found throughout the disclosure or the claims. The subject headings should not be used in construing the scope of the claims or the claim limitations.

Although the technology herein has been described with reference to particular examples, it is to be understood that these examples are merely illustrative of the principles and applications of the technology. In some instances, the terminology and symbols may imply specific details that are not required to practice the technology. For example, although the terms “first” and “second” may be used, unless otherwise specified, they are not intended to indicate any order but may be utilised to distinguish between distinct elements. Furthermore, although process steps in the methodologies may be described or illustrated in an order, such an ordering is not required. Those skilled in the art will recognize that such ordering may be modified and/or aspects thereof may be conducted concurrently or even synchronously.

It is therefore to be understood that numerous modifications may be made to the illustrative examples and that other arrangements may be devised without departing from the spirit and scope of the technology.

### 4.4 REFERENCE SIGNS LIST

| | Reference |
|---|---|
| Item | Number |
| Intra-oral or mandibular advancement device (MAD) | 1000 |
| upper splint | 1100 |
| maxilla/upper gutter portion | 1110 |
| outer edge mandible gutter portion | 1118 |
| maxilla/upper band portion | 1120 |
| upper splint connection point | 1130 |
| second slot | 1132 |
| second obtuse angle | β |
| lower splint | 1200 |
| mandibular/lower gutter portion | 1210 |
| first attachment/rod pin | 1212 |
| arch of the crown of the teeth | 1216 |
| outer edge mandible gutter portion | 1218 |
| mandibular/lower band portion | 1220 |
| top edge | 1222 |
| bottom edge | 1224 |
| lower splint connection point | 1230 |
| first slot | 1232 |
| wing structure | 1240 |
| wing base | 1242 |
| filled portion | 1243 |
| outer side or surface | 1244 |
| elevated portion | 1245 |
| first obtuse angle | α |
| sliding plane surface | 1160, 1260 |
| retention portion/retention area | 1161, 1261 |
| first inner side wall portion | 1162, 1262 |
| second inner side wall portion/undercut portion | 1166, 1266 |
| first joining section | 1164, 1264 |
| inner receiving portion | 1168, 1268 |
| second joining section | 1169, 1269 |
| connecting rod | 1300 |
| first rod end | 1310 |
| first rod pin | 1312 |
| first pin protrusion | 1313 |
| second rod end | 1320 |
| second rod pin | 1322 |
| second pin protrusion | 1323 |
| lower splint | 2100 |
| upper splint | 2200 |
| wing structure | 2240 |
| connecting rods | 2300 |
| first tie rod element | 2300a |
| second tie rod element | 2300b |
| mechanism | 2500 |
| drive system | 2700 |
| sensor(s) | 3600 |
| control system | 3802 |
| memory device | 3804 |
| sensor arrangement | 3806 |
| processor | 3808 |
| external computing device | 3810 |
| I/O device | 3812 |
| power supply | 3814 |
| microphone/speaker | 3816 |
| communication module | 3818 |
| external sensor | 3820 |
| ‘resting’ distance between tie rods | A |
| ‘advanced’ distance between tie rods | B |

1-27. (canceled)

28. An intraoral device system comprising:
an intraoral device including:
an upper splint structured to engage with at least a portion of one or more teeth on a maxilla of a patient;
a lower splint structured to engage with at least a portion of one or more teeth on a mandible of the patient; and
a drive system configured to move the lower splint with respect to the upper splint; and
a feedback system for generating instructions for adjusting the intraoral device, the feedback system including:
a memory storing machine-readable instructions; and
a control system including one or more processors configured to execute the machine-readable instructions to cause the control system to:
receive sensor data from one or more sensors associated with the patient;
process the sensor data to determine a respiratory event likelihood parameter of the patient; and
output control instructions to operate the drive system to move the lower splint with respect to the upper splint responsive to the determined respiratory event likelihood parameter.

29. The intraoral device system of claim 28, wherein the drive system includes a controller and the feedback system is further configured to transmit the control instructions to the controller to operate the drive system.

30. The intraoral device system of claim 28, wherein the drive system is manually adjustable and the control system instructions are outputted as an alert to the patient for manual operation of the drive system.

31. The intraoral device system of claim 28, wherein the sensor data includes intraoral sensor data generated by one or more intraoral sensors associated with the intraoral device, body position data, respiratory sound data and sleep cycle data indicating respective positions of the patient's body, respiratory sounds of the patient and sleep stages of the patient, or any combination thereof, and wherein the control system is configured to determine the respiratory event likelihood parameter based at least in part on the sensor data, where the sensor data includes the intraoral sensor data, the body position data, the respiratory sound data and the sleep cycle data, or any combination thereof.

32. The intraoral device system of claim 28, wherein the control instructions operate the drive system to automatically move the lower splint with respect to the upper splint between an advanced position and a neutral position responsive to the determined respiratory event likelihood parameter.

33. The intraoral device system of claim 28, wherein the determined respiratory event likelihood parameter is correlated with a sleep apnea event, the correlation being used as an indication of the patient's compliance.

34. The intraoral device system of claim 28, further comprising an external computing device configured to communicate with the intraoral device for outputting control instructions for operation of the drive system to move the lower splint with respect to the upper splint responsive to the determined respiratory event likelihood parameter.

35. The intraoral device system of claim 34, wherein the external computing device includes a user interface configured to display an outputted alert, the user interface configured to allow the patient to interact with the manual operation of the drive system.

36. The intraoral device system of claim 28, wherein the respiratory event likelihood parameter has potential values of low, medium and high, and the control instructions are configured to operate the intraoral device in a different way depending on the determined value of the respiratory event likelihood parameter being low, medium, or high.

37. A feedback system for generating instructions for adjusting an intraoral device having an upper splint and a lower splint structured to engage with at least a portion of one or more teeth of a patient and having a drive system for moving the lower splint with respect to the upper splint, the feedback system comprising:
a memory storing machine-readable instructions; and
a control system including one or more processors configured to execute the machine-readable instructions to implement a feedback process to facilitate adjustment of the intraoral device under operation of the drive system by:
receiving sensor data from one or more sensors associated with the patient;
processing the sensor data to determine a respiratory event likelihood parameter of the patient; and
outputting control instructions for operation of the drive system to move the lower splint with respect to the upper splint responsive to the determined respiratory event likelihood parameter.

38. The feedback system of claim 37, wherein the drive system includes a controller and the feedback system is further configured to transmit the control instructions to the controller to operate the drive system.

39. The feedback system of claim 37, wherein the drive system is manually adjustable and the control system instructions are outputted as an alert to the patient for manual operation of the drive system.

40. The feedback system of claim 37, wherein the sensor data includes intraoral sensor data generated by one or more intraoral sensors associated with the intraoral device, body position data, respiratory sound data and sleep cycle data indicating respective positions of the patient's body, respiratory sounds of the patient and sleep stages of the patient, or any combination thereof, and wherein the control system is configured to determine the respiratory event likelihood parameter based at least in part on the sensor data, where the sensor data includes the intraoral sensor data, the body position data, the respiratory sound data and the sleep cycle data, or any combination thereof.

41. The feedback system of claim 37, wherein the control instructions operate the drive system to automatically move the lower splint with respect to the upper splint between an advanced position and a neutral position responsive to the determined respiratory event likelihood parameter.

42. The feedback system of claim 37, wherein the determined respiratory event likelihood parameter is correlated with a sleep apnea event, the correlation being used as an indication of the patient's compliance.

43. The feedback system of claim 37, wherein an external computing device is configured to communicate with the intraoral device for outputting control instructions for operation of the drive system to move the lower splint with respect to the upper splint responsive to the determined respiratory event likelihood parameter.

44. The feedback system of claim 43, wherein the feedback system instructions are outputted as an alert to the patient for operation of the drive system and the external computing device comprises a user interface configured to display the outputted alert, the user interface allowing the patient to interact with the operation of the drive system.

45. The feedback system of claim 37, wherein the respiratory event likelihood parameter has potential values of low, medium and high, and the control instructions are configured to operate the intraoral device in a different way depending on the determined value of the respiratory event likelihood parameter being low, medium, or high.

46. A method for controlling an operation in conjunction with an intraoral device having an upper splint and a lower splint structured to engage with at least a portion of one or more teeth of a patient, the method comprising:
receiving sensor data from one or more sensors associated with the patient;
processing the sensor data to determine a respiratory event likelihood parameter of the patient; and
outputting control instructions for the operation responsive to the respiratory event likelihood parameter.

47. A method according to claim 46, wherein the control instructions operate a drive system of the intraoral device to move the lower splint with respect to the upper splint responsive to the determined respiratory event likelihood parameter.
